(19) United States
(12) Patent Application Publication
(10) Pub. No.: US 2020/0253988 A1
(43) Pub. Date: Aug. 13, 2020

(54) ADJUNCTIVE THERAPY WITH 25-HYDROXYVITAMIN D

(71) Applicant: OPKO IRELAND GLOBAL HOLDINGS, LTD, Grand Cayman (KY)

(72) Inventors: P. Martin Petkovich, Kingston (CA); Joel Z. Melnick, Evanston, IL (US); Jay A. White, Newmarket (CA); Samir P. Tabash, Whitby (CA); Charles W. Bishop, Miami Beach, FL (US); Susan Peers, Toronto (CA); Stephen A. Strugnell, Bay Harbor Islands, FL (US)

(73) Assignee: OPKO IRELAND GLOBAL HOLDINGS, LTD, Grand Cayman (KY)

(21) Appl. No.: 16/859,865

(22) Filed: Apr. 27, 2020

Related U.S. Application Data
(63) Continuation of application No. 16/726,225, filed on Dec. 23, 2019, now abandoned.
Application No. 16/726,225 is a division of application No. 15/502,498, filed on Feb. 7, 2017, now abandoned.
(60) Provisional application No. 62/034,604, filed on Aug. 7, 2014.

(51) Int. Cl. A61K 31/593; A61K 31/663; A61K 9/00; A61K 45/06; A61K 47/14; A61K 9/48; A61K 47/26; A61K 47/38; A61K 47/06; A61K 31/592; A61K 39/395; A61K 31/135; A61K 31/137; A61K 47/44; A61K 31/675

(52) U.S. Cl. CPC A61K 31/593; A61K 2039/505; A61K 9/0053; A61K 45/06; A61K 47/14; A61K 9/4825; A61K 47/26; A61K 47/38; A61K 9/0019; A61K 47/06; A61K 31/592; A61K 9/4875; A61K 39/395; A61K 31/135; A61K 31/137; A61K 47/44; A61K 31/675; C07K 2317/21; C07K 2317/76; A61K 31/663

(57) ABSTRACT

Methods, compositions, and kits for adjunctive therapy using 25-hydroxyvitamin D are disclosed. The 25-hydroxyvitamin D may be administered with an agent that increases the risk of hypocalcemia and/or an anticancer agent. The adjunctive therapy is effective to treat and prevent iatrogenic hypocalcemia and/or secondary hyperparathyroidism, as well as delay cancer progression and the time to a post-treatment skeletal related event.

### CROSS-REFERENCE TO RELATED APPLICATION

The benefit under 35 U.S.C. § 119(e) of U.S. Provisional Patent Application Ser. No. 62/034,604 filed Aug. 7, 2014, is hereby claimed, and the disclosure thereof is incorporated herein by reference.

### BACKGROUND

The Vitamin D metabolites known as 25-hydroxyvitamin D₂ and 25-hydroxyvitamin D₃ (collectively referred to as “25-hydroxyvitamin D”) are Vitamin D prohormones that contribute to the maintenance of adequate levels of Vitamin D hormones, calcium and phosphorus in the bloodstream. The prohormone 25-hydroxyvitamin D₂ is produced from Vitamin D₂ (ergocalciferol), and 25-hydroxyvitamin D₃ (calcifediol) is produced from Vitamin D₃ (cholecalciferol), primarily by one or more enzymes located in the liver. The two prohormones also can be produced outside of the liver from Vitamin D₂ and Vitamin D₃ (collectively referred to as “Vitamin D”) in certain cells, such as enterocytes, which contain enzymes identical or similar to those found in the liver.

The Vitamin D prohormones are further metabolized in the kidneys by the 1α-hydroxylase enzyme CYP27B 1 into potent hormones. The prohormone 25-hydroxyvitamin D₂ is metabolized into a hormone known as 1α,25-dihydroxyvitamin D₂ (ercalcitriol); likewise, 25-hydroxyvitamin D₃ is metabolized into 1α,25-dihydroxyvitamin D₃ (calcitriol). Production of these hormones from the prohormones also can occur outside of the kidney in cells which contain the required enzyme(s).

The Vitamin D hormones have essential roles in human health which are mediated by intracellular Vitamin D receptors (VDR). The Vitamin D hormones participate in the regulation of cellular differentiation and growth, parathyroid hormone (PTH) secretion by the parathyroid glands, and normal bone formation and metabolism. In particular, the Vitamin D hormones regulate blood calcium levels by controlling the absorption of dietary calcium and phosphorus by the small intestine and the reabsorption of calcium by the kidneys. Under normal conditions, actions of Vitamin D on stimulating intestinal calcium absorption predominate, such that dietary calcium is the main source of serum calcium. However if dietary calcium or vitamin D is insufficient, the parathyroid gland increases secretion of PTH to enhance calcium mobilization from bone to maintain serum calcium levels. Excessive hormone levels, whether transient or prolonged, can lead to abnormally elevated urine calcium (hypercalciuria), blood calcium (hypercalcemia) and blood phosphorus (hyperphosphatemia). Insufficient hormone levels can lead to the opposite syndrome of abnormally low blood calcium levels (hypocalcemia). Vitamin D hormones are also required for the normal functioning of the musculoskeletal, immune and renin-angiotensin systems. Numerous other roles for Vitamin D hormones are being postulated and elucidated, based on the documented presence of intracellular VDR in nearly every human tissue.

Left untreated, inadequate Vitamin D supply can cause serious bone disorders, including rickets and osteomalacia, and may contribute to the development of many other disorders including osteoporosis, non-traumatic fractures of the spine and hip, obesity, diabetes, muscle weakness, immune deficiencies, hypertension, psoriasis, and various cancers.

The Institute of Medicine (IOM) of the National Academy of Sciences has concluded that an Adequate Intake (AI) of Vitamin D for a healthy individual ranges from 200 to 600 IU per day, depending on the individual's age and sex (Standing Committee on the Scientific Evaluation of Dietary Reference Intakes, Dietary reference intakes: calcium, phosphorus, magnesium, vitamin D, and fluoride. Washington, D.C.: National Academy Press (1997), incorporated by reference). The AI for Vitamin D was defined primarily on the basis of a serum 25-hydroxyvitamin D level sufficient to prevent Vitamin D deficiency rickets or osteomalacia (or greater than or equal to 11 ng/mL). The IOM also established a Tolerable Upper Intake Level (UL) for Vitamin D of 2,000 IU per day, based on evidence that higher doses are associated with an increased risk of hypercalciuria, hypercalcemia and related sequelae, including cardiac arrhythmias, seizures, and generalized vascular and other soft-tissue calcification.

Currently available oral Vitamin D supplements are far from ideal for achieving and maintaining optimal blood 25-hydroxyvitamin D levels. These preparations typically contain 400 IU to 5,000 IU of Vitamin D₃ or 50,000 IU of Vitamin D₂ and are formulated for quick or immediate release in the gastrointestinal tract. When administered at chronically high doses, as is often required for Vitamin D repletion, these products have significant, and often severe, limitations.

Abnormalities of Vitamin D signaling and metabolism exist in a wide variety of tumors (Krishnan et al., (2012). Rheum Dis Clin North Am 38, 161-178) and are thought to be due to increased expression of CYP24 (Luo et al., (2013) J Steroid Biochem Mol Biol 136, 252-257). Cancer patients generally exhibit vitamin D insufficiency, therefore, calcium resorption from bone calcium stores plays a dominant role in the normalization of blood calcium levels. Regardless of the cancer type, low serum levels of 25-hydroxyvitamin D and decreased VDR activation have been associated with increased metastasis. Cancer mortality is usually a consequence of metastasis. For certain types of cancer, notably breast and prostate, the bulk of tumor burden at the time of death is in bone. The impact of metastasis on bone metabolism and consequent morbidity is considerable and, depending on the origin of the primary tumor, is either osteolytic (e.g., breast, myeloma) or osteoblastic (e.g., prostate) in nature. However, since bone formation and bone resorption are coupled, “osteolytic” and “osteoblastic” categorizations correspond to the net balance of bone metabolism associated with metastases. A number of factors released from tumors can affect net balance of bone metabolism, including parathyroid hormone related peptide (PTHrP), transforming growth factor-β (TGF-β), insulin-like growth factors (IGF), bone morphogenetic factors (BMP) and platelet-derived growth factors (PDGF).

PTHrP is produced by certain types of cancer cells, such as breast, and can trigger net bone resorption by stimulating the production of the ligand for the receptor activator of NFκB (RANKL) (Rabbani, S. A. (2000). Int J Oncol 16, 197-206; Soyfoo et al. (2013). Support Care Cancer 21, 1415-1419). Like PTH, PTHrP can be regulated by activating the Vitamin D signaling pathway (Bhatia et al. (2009). Mol Cancer Ther 8, 1787-1798; El Abdaimi et al. (1999). Cancer Res 59, 3325-3328.). Consequently, the use of Vitamin D and related analogs has been proposed to help control excessive hypercalcemia caused by PTHrP overexpression in breast and prostate cancers (Richard et al. (2005) Crit Rev Eukaryot Gene Expr 15, 115-132.). The majority of instances of hypercalcemia in cancer patients are thought to be related to the production of PTHrP (Motellon et al. (2000) Clin Chim Acta 290, 189-197.). In some cases, hypercalcemia of malignancies has been associated with the use of Vitamin D or calcifediol and is related to elevated PTHrP expression. Like PTH, PTHrP expression can increase expression of CYP27B 1, the kidney enzyme responsible for activating calcifediol. Therefore, a cancer patient with vitamin D insufficiency and higher than normal levels of PTHrP could potentially express increased levels of unoccupied CYP27B 1; a sudden bolus of calcifediol could cause a surge in 1,25-dihydroxyvitamin D and potentially result in hypercalcemic episodes (Motellon et al 2000, supra; Sato et al. (1993). Intern Med 32, 886-890.) and further upregulation of CYP24. These hypercalcemic episodes, in contrast to those caused by PTHrP stimulation of RANKL, are due to increased rate of intestinal absorption of Ca.

The relationship between the progression of tumor metastases and bone catabolism is determined to a large extent on the tumor microenvironment within bone. In certain types of cancers, such as prostate cancer, bone formation can be stimulated by TGF-β, IGFs, PDGF and BMPs and these factors play an important role in establishing the bone microenvironment. These patients can suffer from hypocalcemia, which is the reduction of serum calcium levels in the blood. Severe hypocalcemia is sometimes referred to as “hungry bone” syndrome. Accordingly, the state of bone health may be an important determinant of the progression of the metastatic process, including the tumor cell invasion of bone, the angiogenic response, and tumor cell proliferation, as well as differentiation of bone cell precursors into osteoblasts and osteoclasts. There is evidence that vitamin D status may have an influence on each of these parameters, suggesting that vitamin D adequacy may be essential to minimize the progression of bone metastases. Although numerous clinical studies have attempted to raise Vitamin D levels for the treatment of various cancers, currently available therapies do not safely raise 25-hydroxyvitamin D levels high enough to establish the impact 25-hydroxyvitamin D has on tumor growth and metastasis or associated morbidities.

Because bone resorption is a common pathophysiology of bone metastases regardless of primary tumor type, patients are typically treated with bone antiresorptive agents, which inhibit bone resorption by targeting bone osteoclasts to decrease their osteolytic activity. Antiresorptive therapies, also known as bone-sparing agents, reduce the impact of cancer-related increases in bone resorption. Antiresorptive agents can prevent or delay skeletal related events (SRE). SRE are defined as pathological fractures, radiation or surgery to bone, and spinal cord compression, and are used to evaluate the clinical efficacy of antiresorptive agents because SRE are associated with poor prognosis and quality of life. Because antiresorptive agents can slow bone loss, they are also prescribed for patients with osteoporosis and other bone disorders. Examples of antiresorptive agents include bisphosphonates such as zoledronic acid, selective estrogen receptor modulators (SERMs), calcitonin, estrogen, and monoclonal antibodies such as denosumab. Treatment with antiresorptive agents also reduces the efficiency of PTH-stimulated resorption of bone, thus patients must rely on intestinal absorption of calcium for maintaining serum calcium levels.

One of the most important and immediate side effects of antiresorptive agents is hypocalcemia. Other therapeutic agents that can increase the risk of hypocalcemia include anticonvulsant agents, corticosteroids, antihypercalcemia agents, antimicrobial agents, and combinations thereof. Serum calcium is critical for the normal function of nerves and muscles in the body, and serum calcium levels are tightly regulated within narrow limits in healthy subjects. Hypocalcemia can be a significant source of morbidity and mortality. Severe hypocalcemia, in which serum calcium levels are reduced to below the lower limit of normal, can result in life-threatening consequences, including muscle tetany and cardiac arrest. Such treatment-induced, also known as iatrogenic, hypocalcemia, can be serious, even fatal, and therefore must be controlled.

Following administration of the antiresorptive agent denosumab, hypocalcemia is believed to result directly from the inhibitory effects of denosumab on the activity and numbers of bone-resorbing osteoclastic bone cells. Clinical studies have suggested reduced levels of calcium in the blood as soon as one day after initiation of denosumab treatment. Similarly, in a recent study of patients with bone metastases treated with the antiresorptive agent zoledronic acid, 39% of the patients developed hypocalcemia (Zuradelli et al., (2009) Oncologist 14, 548-556). Hypocalcemia is one of the most common adverse reactions resulting in discontinuation of therapy with zoledronic acid or denosumab.

Vitamin D supplementation is therefore recommended for patients on antiresorptive therapy. The treatment protocols in published repeat-dose clinical studies for denosumab have uniformly called for denosumab-treated subjects to receive daily supplements of calcium (0.5 to 1.0 g or more) and at least 400 to 800 IU vitamin D (cholecalciferol and/or ergocalciferol) in order to prevent hypocalcemia. Recommendations for calcium and vitamin D supplementation of denosumab-treated subjects have been included in the FDA-approved labeling for denosumab. However, currently available oral vitamin D supplements are not optimal for increasing and maintaining serum levels of either 25-hydroxyvitamin D or 1,25-dihydroxyvitamin D at desirable levels. The inadequacy of currently available vitamin D supplements at completely mitigating hypocalcemia in denosumab-treated subjects is highlighted by a recent Advisory from Health Canada, which noted that postmarketing cases of severe symptomatic hypocalcemia have occurred in denosumab-treated subjects at an estimated rate of 1 to 2%, including some cases that were fatal.

Another side effect of antiresorptive agents and other agents that increase the risk of hypocalcemia is secondary hyperparathyroidism (SHPT). Decreases in serum calcium can result in increased production of PTH. Elevated PTH levels are common in patients undergoing treatment with antiresorptive agents, indicating an increased vitamin D requirement. Regulation of blood calcium requires adequate production of calcitriol, which stimulates intestinal absorption of dietary calcium and reabsorption of calcium by the kidney. Calcitriol, in concert with elevated PTH, also mobilizes calcium from bone. Adequate calcitriol production requires a sufficient supply of the precursor, calcifediol, and the first sign of inadequate calcitriol production is an increase in plasma PTH. PTH stimulates expression of CYP27B 1 in the kidney and, thereby, increases conversion of calcifediol to calcitriol. When serum calcitriol levels are restored to adequate levels, PTH secretion decreases. If serum calcitriol levels cannot be corrected, as in the case of a calcifediol supply shortage (i.e., vitamin D insufficiency), plasma PTH remains elevated causing continuous mobilization of calcium from bone. A recent study (Berruti et al. (2012) Oncologist 17, 645-652) reported that 82% to 90% of subjects with prostate cancer metastatic to bone and receiving zoledronic acid exhibited elevated PTH, compared to 17% of patients receiving placebo. The elevated PTH was negatively associated with survival. The prevalence and persistence of SHPT in patients on antiresorptive therapies even though supplemented with Vitamin D and calcium indicates that appropriate supplementation regimens have not yet been clearly defined for this patient population, and the efficacy of antiresorptive agents can be limited by even mild hypocalcemia and/or SHPT.

Clearly, an alternative approach to currently available Vitamin D supplementation is needed in patients with cancer and in patients treated with an agent that increases the risk of hypocalcemia.

### SUMMARY

The present disclosure relates to 25-hydroxyvitamin D therapy as adjunctive therapy and/or to treat cancer in a patient. In one aspect, a method of treating or preventing iatrogenic hypocalcemia and/or secondary hyperparathyroidism in a patient treated with an agent that increases the risk of hypocalcemia comprises administering to the patient an effective amount of 25-hydroxyvitamin D. In another aspect, a method of increasing bone mineral density in a patient treated with an agent that increases the risk of hypocalcemia comprises administering to the patient an effective amount of 25-hydroxyvitamin D.

In another aspect, a method of decreasing the blood level of a bone resorption marker in a patient treated with an agent that increases the risk of hypocalcemia comprises administering to the patient an effective amount of 25-hydroxyvitamin D. In another aspect, a method of treating bone pain in a patient treated with an agent that increases the risk of hypocalcemia comprises administering to the patient an effective amount of 25-hydroxyvitamin D.

In another aspect, a method of increasing the time to the first post-treatment skeletal-related event in a patient treated with an agent that increases the risk of hypocalcemia comprises administering to the patient an effective amount of 25-hydroxyvitamin D. In another aspect, a method of treating a patient treated with an agent that increases the risk of hypocalcemia comprises administering to the patient an effective amount of 25-hydroxyvitamin D to effectively and safely restore blood 25-hydroxyvitamin D levels to at least 30 ng/mL and to maintain blood 25-hydroxyvitamin D levels at such optimal levels.

In any of the methods disclosed herein, the agent that increases the risk of hypocalcemia is optionally selected from the group consisting of an antiresorptive agent, an anti-convulsant agent, a corticosteroid, an anti-hypercalcemia agent, an antimicrobial agent, and combinations thereof. In one aspect, the agent that increases the risk of hypocalcemia is an antiresorptive agent, optionally selected from the group consisting of bisphosphonates (e.g., zoledronic acid, alendronate, risedronate, ibandronate, etidronate, and pamidronate), selective estrogen receptor modulators (e.g., raloxifene), calcitonin, hormones (e.g., estrogen), and monoclonal antibodies (e.g., denosumab).

In another aspect, a method of lowering elevated serum parathyroid hormone levels in a patient having a bone metastasis and treated with an antiresorptive agent comprises administering an effective amount of 25-hydroxyvitamin D. In another aspect, a method of stabilizing serum calcium levels in a patient having a bone metastasis and treated with an antiresorptive agent comprises administering an effective amount of 25-hydroxyvitamin D. In still another aspect, a method of treating hungry bone syndrome comprises administering an effective amount of 25-hydroxyvitamin D to a patient in need of thereof.

In any of the methods of the present disclosure, the patient optionally has osteoporosis and/or cancer. In one aspect, a method of managing iatrogenic hypocalcemia and secondary hyperparathyroidism in a patient with a bone metastasis treated with an antiresorptive agent comprises administering an effective amount of 25-hydroxyvitamin D to prevent or reverse the iatrogenic hypocalcemia and lower the patient's serum parathyroid hormone level. In another aspect, a method of mitigating cancer progression and/or a skeletal related event in a patient with a bone tumor, optionally a bone metastasis from a solid tumor, comprises treating the patient with (a) an anticancer agent; (b) an antiresorptive agent; and (c) a 25-hydroxyvitamin D compound, wherein the combination of (a), (b), and (c) is effective to slow tumor growth and/or metastasis and/or increase the time to the first post-treatment skeletal-related event. In still another aspect, a method of treating a patient having cancer and a bone metastasis comprises the administration of (a) a prophylactic and continuing course of an effective amount of 25-hydroxyvitamin D to stabilize 25-hydroxyvitamin D levels and calcium levels in the patient without causing or exacerbating hypercalcemia; followed by (b) treatment with an agent known to increase the risk of iatrogenic hypocalcemia, wherein the treatment in step (a) prevents and/or treats the iatrogenic hypocalcemia in the patient.

In another aspect, a method of mitigating the progression of cancer in the bone in a patient comprises administering an effective amount of 25-hydroxyvitamin D. In another aspect, a method of inhibiting the proliferation and migration of cancer cells comprises administering an effective amount of 25-hydroxyvitamin D to a patient in need thereof. In another aspect, a method of treating cancer in a patient comprises administering to the patient an effective amount of a combination of 25-hydroxyvitamin D and an anticancer agent. In any of the foregoing methods, the patient optionally has a cancer selected from the group consisting of bone cancer, bladder cancer, breast cancer, colon cancer, endometrial cancer, kidney cancer, leukemia, lung cancer, lymphoma, pancreatic cancer, prostate cancer, skin cancer, thyroid cancer, and metastatic forms thereof.

The present disclosure also relates to the use of 25-hydroxyvitamin D, optionally in a modified release formulation, as adjunctive therapy to treat hypocalcemia in a patient in need thereof. In one aspect, the disclosure provides a pharmaceutical composition comprising (a) 25-hydroxyvitamin D and (b) an agent that increases the risk of hypocalcemia and/or an anticancer agent. In another aspect, the disclosure provides a kit comprising (a) 25-hydroxyvitamin D; (b) an agent that increases the risk of hypocalcemia and/or an anticancer agent; and (c) instructions for co-administering effective amounts of (a) and (b) to a patient in need thereof.

In another aspect, a method according to the present disclosure comprises administering 25-hydroxyvitamin D in a modified release formulation, optionally an oral modified release formulation. In another aspect, the 25-hydroxyvitamin D is administered in a sterile intravenous formulation. In various aspects, the 25-hydroxyvitamin D can be selected from the group consisting of 25-hydroxyvitamin D₂, 25-hydroxyvitamin D₃, 25-hydroxyvitamin D₄, 25-hydroxyvitamin Ds, 25-hydroxyvitamin D₇ and combinations thereof.

For the compositions and methods described herein, optional features, including but not limited to components, compositional ranges thereof, substituents, conditions, and steps, are contemplated to be selected from the various aspects, embodiments, and examples provided herein.

Further aspects and advantages will be apparent to those of ordinary skill in the art from a review of the following detailed description, taken in conjunction with the drawings. While the compositions and methods are susceptible of embodiments in various forms, the description hereafter includes specific embodiments with the understanding that the disclosure is illustrative and is not intended to limit the invention to the specific embodiments described herein.

### DETAILED DESCRIPTION

The present disclosure relates to 25-hydroxyvitamin D therapy as adjunctive therapy and in the treatment of cancer. In various embodiments, the disclosure provides methods for dosing a subject receiving treatment with an agent that increases the risk of hypocalcemia and/or an anticancer agent with an effective amount of 25-hydroxyvitamin D, optionally as a modified release oral formulation or administered in intravenous form. The administration of 25-hydroxyvitamin to a patient according to the present disclosure effectively achieves one or more of the following: (a) treats or prevents hypocalcemia, e.g., iatrogenic hypocalcemia; (2) treats or prevents secondary hyperparathyroidism; (3) increases bone mineral density; (4) decreases the blood level of a bone resorption marker; (5) decreases bone pain; (6) increases the time to the first post-treatment skeletal related event; (6) safely restores blood 25-hydroxyvitamin D levels to optimal levels (defined for human subjects as greater than 30 ng/mL) and maintains blood 25-hydroxyvitamin D levels at such optimal levels without causing hypocalcemia or hypercalcemia; (7) lowers elevated serum parathyroid hormone levels; (8) stabilizes serum calcium levels; (9) treats hungry bone syndrome; (10) manages iatrogenic hypocalcemia and secondary hyperparathyroidism in a patient with a bone tumor; (11) mitigates cancer progression, i.e., by inhibiting the proliferation and/or migration of cancer cells; (12) restores or maintains serum calcium levels to at least 8.0 mg/dL, optionally at least 8.3 mg/dL or 8.5 mg/dL, further optionally up to 11.6 mg/dL, e.g. in a range of 8.3 mg/dL and 11.6 mg/dL, corrected for serum albumin; (13) safely increases serum levels of 1,25-dihydroxyvitamin D, optionally to at least 50 pg/mL; (14) achieves or maintains safe serum phosphorus levels and prevents or treats hypophosphatemia; (15) has a positive effect on the serum level of a marker of bone formation; and/or (16) maintains or decreases tumor burden.

The present disclosure also relates to the use of 25-hydroxyvitamin D as adjunctive therapy to treat hypocalcemia, and compositions and kits comprising (a) 25-hydroxyvitamin D and (b) an agent that causes hypocalcemia and/or an anticancer agent.

The methods, compositions, and kits of the present disclosure are contemplated to include embodiments including any combination of one or more of the additional optional elements, features, and steps further described below, unless stated otherwise.

In jurisdictions that forbid the patenting of methods that are practiced on the human body, the meaning of “administering” a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, “administering” compositions includes both methods practiced on the human body and also the foregoing activities.

As used herein, the following definitions may be useful in aiding the skilled practitioner in understanding the invention:

As used herein, the term “comprising” indicates the potential inclusion of other agents, elements, steps, or features, in addition to those specified.

As used herein, the term “25-hydroxyvitamin D” refers to one or more of 25-hydroxyvitamin D₂, 25-hydroxyvitamin D₃, 25-hydroxyvitamin D₄, 25-hydroxyvitamin Ds, 25-hydroxyvitamin D₇, analogs of the foregoing, and combinations thereof. It is specifically contemplated that in any embodiment described herein, 25-hydroxyvitamin D can include 25-hydroxyvitamin D₃, 25-hydroxyvitamin D₂, or a combination of 25-hydroxyvitamin D₃ and 25-hydroxyvitamin D₂. For example, it is specifically contemplated that in any embodiment described herein, 25-hydroxyvitamin D can include 25-hydroxyvitamin D₃. Serum total 25-hydroxyvitamin D refers to the total of all such 25-hydroxyvitamin D forms measured by assay, unless a particular 25-hydroxyvitamin D form is referred to.

As used herein, the term “1,25-dihydroxyvitamin D” refers to one or more of 1,25-dihydroxyvitamin D₂, 1,25-dihydroxyvitamin D₃, 1,25-dihydroxyvitamin D₄, 1,25-dihydroxyvitamin Ds, 1,25-dihydroxyvitamin D₇, analogs of the foregoing, and combinations thereof. For example, 1,25-dihydroxyvitamin D can include 1,25-dihydroxyvitamin D₂, 1,25-dihydroxyvitamin D₃, or a combination of 1,25-dihydroxyvitamin D₂ and 1,25-dihydroxyvitamin D₃. Serum total 1,25-dihydroxyvitamin D will be understood to refer to the total of all such 1,25-dihydroxyvitamin D forms by assay, unless a reference is made to a particular 1,25-dihydroxyvitamin D form.

As used herein, the term “adjunctive therapy” refers to administration of 25-hydroxyvitamin D to a patient who is (a) currently receiving; (b) has previously received; or (c) will receive, treatment with a therapeutic agent that is not 25-hydroxyvitamin D. In one aspect, adjunctive therapy refers to the administration of 25-hydroxyvitamin D to a patient before administration with the therapeutic agent that is not 25-hydroxyvitamin D. In another aspect, adjunctive therapy refers to the administration of 25-hydroxyvitamin D to a patient concomitant with administration with the therapeutic agent that is not 25-hydroxyvitamin D. In another aspect, adjunctive therapy refers to the administration of 25-hydroxyvitamin D to a patient after administration with the therapeutic agent that is not 25-hydroxyvitamin D. The therapeutic agent that is not 25-hydroxyvitamin D is optionally an agent that increases the risk of hypocalcemia or an anticancer agent.

As used herein, the term “antiresorptive agent” refers to a compound that inhibits bone resorption, i.e., a “bone-sparing” agent. Examples of antiresorptive agents include, but are not limited to, bisphosphonates (e.g., zoledronic acid, alendronate, risedronate, ibandronate, etidronate, and pamidronate), selective estrogen receptor modulators (e.g., raloxifene), calcitonin, hormones (e.g., estrogen), and monoclonal antibodies (e.g., denosumab).

As used herein, the term “co-administer” refers to administering an agent that increases the risk of hypocalcemia or an anticancer agent and 25-hydroxyvitamin D to a subject in a manner that permits the agents to exert their respective pharmacological effects during an overlapping period of time and is a form of adjunctive therapy. The co-administered agent and 25-hydroxyvitamin D can be administered by the same or different routes, and in the same or different compositions. The co-administered agent and 25-hydroxyvitamin D can be administered at the same time, or at different times during a course of treatment (e.g., on alternating days or at different times in the same day). For example, it is contemplated that co-administration can include administration of both an antiresorptive agent and a 25-hydroxyvitamin D compound within six months or less of each other, or within three months or less of each other, or within one month or less of each other, or within two weeks or less of each other, or within one week or less of each other, or within two days or less of each other, or on the same day. A course of the agent that increases the risk of hypocalcemia or an anticancer agent can include a relatively longer dose interval, e.g., every six months, while 25-hydroxyvitamin D treatment can be on a shorter interval, e.g., daily.

As used herein, the term “substantially constant” with respect to the serum or blood level of 25-hydroxyvitamin D means that the release profile of any formulation administered as detailed herein should not include transient increases in total serum or blood levels of 25-hydroxyvitamin D₃ or 25-hydroxyvitamin D₂ of greater than approximately 3 ng/mL after administration of a unit dose.

As used herein, the term “modified release” refers to any modification of release from an immediate release profile and can include controlled or sustained release and/or delayed release characteristics. As used herein, the term “controlled release” and “sustained release” are used interchangeably and refer to the release of the administered 25-hydroxyvitamin D from a composition for an extended period of time, e.g., 4 to 24 hours or even longer.

As used herein, the term “Vitamin D toxicity” refers to the side effects associated with excessive administration of 25-hydroxyvitamin D and excessively elevated 25-hydroxyvitamin D blood levels, including, but not limited to, nausea, vomiting, polyuria, hypercalciuria, hypercalcemia and hyperphosphatemia.

As used herein, the term “hypocalcemia” refers to a condition wherein a patient has a corrected serum levels of calcium below about 8.3 mg/dL or below about 8.5 mg/dL. Severe hypocalcemia refers to a condition wherein the patient has a corrected serum level of calcium below about 7 mg/dL. Normal and safe corrected serum levels of calcium for a human are in a range of about 8.3 to about 11.6 mg/dL. Corrected serum levels of calcium refer to values corrected for serum albumin less than 4.0 g/dL. The term “iatrogenic hypocalcemia” refers to hypocalcemia that occurs following treatment with a therapeutic agent, i.e., an agent that increases the risk of hypocalcemia. Examples of agents that increase the risk of hypocalcemia include, but are not limited to, antiresorptive agents, anticonvulsant agents, corticosteroids, antihypercalcemia agents, antimicrobial agents, and combinations thereof.

As used herein, the term “hypercalcemia” refers to a condition in a patient wherein the patient has corrected serum levels of calcium above about 11.6 mg/dL.

As used herein, the term “hypophosphatemia” refers to a condition wherein a patient has a serum phosphorous level below about 2.5 mg/dL. Normal and safe values for serum phosphorous in a human are in a range of about 2.5 mg/dL to about 4.5 mg/dL.

As used herein, the term “hyperphosphatemia” refers to a condition in a patient wherein the patient has serum phosphorous levels above about 4.5 mg/dL.

As used herein, the term “supraphysiologic” in reference to intralumenal, intracellular and/or blood concentrations of 25-hydroxyvitamin D refers to a combined concentration of 25-hydroxyvitamin D forms during a 24-hour post-dose period which is more than 5 ng/mL greater than the generally stable levels observed over the course of the preceding 24-hour period by laboratory measurement. “Supraphysiologic” in reference to intralumenal, intracellular and/or blood concentrations of 1,25-dihydroxyvitamin D refers to a combined concentration of 1,25-dihydroxyvitamin D forms more than 5 pg/mL greater than the generally stable levels observed over the course of the preceding 24-hour period by laboratory measurement.

As used herein, the term “Vitamin D insufficiency and deficiency” is generally defined in humans as having a serum 25-hydroxyvitamin D level below 30 ng/mL (National Kidney Foundation guidelines, NKF, Am. J. Kidney Dis. 42:S1-S202 (2003), incorporated herein by reference).

It is specifically understood that any numerical value recited herein includes all values from the lower value to the upper value, i.e., all possible combinations of numerical values between the lowest value and the highest value enumerated are to be considered to be expressly stated in this application. For example, if a concentration range or a beneficial effect range is stated as 1% to 50%, it is intended that values such as 2% to 40%, 10% to 30%, or 1% to 3%, etc., are expressly enumerated in this specification. These are only examples of what is specifically intended.

In one aspect, the disclosure provides methods of adjunctive therapy using 25-hydroxyvitamin D is patients treated with an agent that increases the risk of hypocalcemia and/or an anticancer agent. The disclosed methods provide dual unexpected benefits with continued regular administration over a prolonged period of time of unsurpassed effectiveness in restoring blood 25-hydroxyvitamin D to optimal levels and unsurpassed safety relative to currently available formulations of Vitamin D or 25-hydroxyvitamin D. The methods of the present disclosure can include providing a gradual, sustained and direct release of an effective amount of 25-hydroxyvitamin D, preferentially to circulating DBP (rather than to chylomicrons), such that blood, intralumenal and intracellular 25-hydroxyvitamin D concentration spikes, and related unwanted catabolism are mitigated or eliminated. Administration of 25-hydroxyvitamin D according to the present disclosure enhances the intestinal absorption of calcium and reduces PTH-mediated bone resorption. This reduces the likelihood of hypocalcemic events and at the same time, reduces the expression of PTH, thereby mitigating the metastatic impact on resorption of bone. Raising 25-hydroxyvitamin levels in patients as described herein can stabilize serum calcium levels and have an impact on bone microenvironment, cancer progression, and skeletal related events.

Adjunctive therapy comprising 25-hydroxyvitamin D according to the present disclosure improves the efficacy of a co-administered agent that increases the risk of hypocalcemia (e.g., an antiresorptive agent) by one or more measures. In one embodiment, co-administering an agent that increases the risk of hypocalcemia and an effective amount of 25-hydroxyvitamin D is effect to treat or prevent iatrogenic hypocalcemia and SHPT. In another embodiment, co-administering an agent that increases the risk of hypocalcemia and an effective amount of 25-hydroxyvitamin D is effective to increase bone mineral density. In another embodiment, co-administering an agent that increases the risk of hypocalcemia and an effective amount of 25-hydroxyvitamin D is effect to decrease bone pain. In another embodiment, co-administering an agent that increases the risk of hypocalcemia and an effective amount of 25-hydroxyvitamin D is effective to treat secondary hyperparathyroidism by lowering elevated plasma PTH levels, optionally by at least 30%. In another embodiment, co-administering an agent that increases the risk of hypocalcemia and an effective amount of 25-hydroxyvitamin D is effective to decrease the incidence or risk of hypocalcemia. In another embodiment, co-administering an agent that increases the risk of hypocalcemia and an effective amount of 25-hydroxyvitamin D is effective to stabilize serum calcium levels, optionally at a level in a range of 8.3 mg/dL and 11.6 mg/dL, corrected for serum albumin. In another embodiment, co-administering an agent that increases the risk of hypocalcemia and an effective amount of 25-hydroxyvitamin D is effective to increase blood levels of a bone formation marker. In another embodiment, co-administering an agent that increases the risk of hypocalcemia and an effective amount of 25-hydroxyvitamin D is effective to decrease blood levels of a bone resorption marker. In another embodiment, co-administering an agent that increases the risk of hypocalcemia and an effective amount of 25-hydroxyvitamin D is effective to delay the time to the first post-treatment SRE. In another embodiment, co-administering an agent that increases the risk of hypocalcemia and an effective amount of 25-hydroxyvitamin D is effective to delay the time to further bone metastasis. In another embodiment, co-administering an agent that increases the risk of hypocalcemia and an effective amount of 25-hydroxyvitamin D is effective to safely increase serum total 25-hydroxyvitamin D levels to at least 30 ng/mL, optionally to supraphysiologic levels. In another embodiment, co-administering an agent that increases the risk of hypocalcemia and an effective amount of 25-hydroxyvitamin D is effective to safely increase serum total 1,25-hydroxyvitamin D levels, optionally to supraphysiologic levels. In another embodiment, co-administering an agent that increases the risk of hypocalcemia and an effective amount of 25-hydroxyvitamin D will be effective to attenuate or halt cancer progression, e.g., by inhibiting the proliferation and migration of cancer cells or maintaining or decreasing tumor burden.

In one embodiment, an effective amount of 25-hydroxyvitamin D is administered to a patient that is receiving or has previously received treatment with an agent that increases the risk of hypocalcemia. For example, in one embodiment, 25-hydroxyvitamin D is administered following administration of an agent that increases the risk of hypocalcemia, e.g., an antiresorptive agent or antihypercalcemia agent. In another embodiment, 25-hydroxyvitamin D is administered prophylactically to a patient before treatment with an agent that increases the risk of hypocalcemia is undertaken. In various embodiments, the agent that increases the risk of hypocalcemia is optionally selected from the group consisting of an antiresorptive agent, an anticonvulsant agent, a corticosteroid, an antihypercalcemia agent, an antimicrobial agent, and combinations thereof. For example, in one embodiment, the agent that increases the risk of hypocalcemia is an antihypercalcemia agent, such as cinacalcet (SENSIPAR, Amgen Inc., Thousand Oaks, Calif.). In another embodiment, the agent that increases the risk of hypocalcemia is an antiresorptive agent, optionally selected from the group consisting of bisphosphonates (e.g., zoledronic acid), RANKL inhibitors (e.g., denosumab), monoclonal antibodies (e.g., denosumab), and combinations thereof.

Another aspect of the present disclosure is treatment of cancer in a patient. Most cancer patients exhibit vitamin D insufficiency (i.e., serum total 25-hydroxyvitamin D less than 30 ng/mL). Although there are a number of possible causes, including diet and reduced exposure to sunlight, recent evidence suggests that accelerated vitamin D catabolism may also be a contributor. Genome amplification at the 20q.13 chromosomal locus that encodes CYP24A1 (Albertson et al. (2000) Nat Genet 25, 144-146) has been identified in a number of tumor types (Krishnan et al., supra). Overexpression of CYP24A1 mRNA is reported in a wide variety of human cancers, including breast (Friedrich et al. (2003) Recent Results Cancer Res 164, 239-246), lung (Parise et al. (2006) Int J Cancer 119, 1819-1828) and colorectal, and in some cases, is linked to a poor prognosis and overall reduced survival (Mimori et al. (2004) Ann Oncol 15, 236-241). Overexpression of CYP24A1 increases the growth potential of tumor cells and lowers the responsiveness of tumors to the anti-cancer effects of endogenous calcitriol (Anderson et al. (2006) Cancer Chemother Pharmacol 57, 234-240; Friedrich et al., supra). Higher levels of 25-hydroxyvitamin D may therefore be required to achieve vitamin D adequacy for normal cellular and physiological functions and to exert optimal antitumor effects. Administration of 25-hydroxyvitamin D as described herein acts through activation of the Vitamin D receptor pathway to maintain normal calcium homeostasis and can thereby target a variety of tumor types.

Administration of 25-hydroxyvitamin D to a patient having cancer and adjunctive therapy comprising 25-hydroxyvitamin D and an anticancer agent is contemplated to have a therapeutic effect by one or more measures. In one embodiment, administering an effective amount of 25-hydroxyvitamin D, optionally with an anticancer agent and/or agent that increases the risk of hypocalcemia, to the patient is effective to treat cancer, e.g., by inhibiting the proliferation and migration of cancer cells. In another embodiment, administering an effective amount of 25-hydroxyvitamin D, optionally with an anticancer agent and/or agent that increases the risk of hypocalcemia, is effective to maintain or decrease the patient's tumor burden. In another embodiment, administering an effective amount of 25-hydroxyvitamin D, optionally with an anticancer agent and/or agent that increases the risk of hypocalcemia, is effective to mitigate the progression of cancer in the bone. In another embodiment, administering an effective amount of 25-hydroxyvitamin D, optionally with an anticancer agent and/or agent that increases the risk of hypocalcemia, is effective to slow tumor growth and/or metastasis and increase the time to the first-post-treatment SRE in a patient with a bone tumor, optionally a bone metastasis from a solid tumor. In another embodiment, administration of a prophylactic and continuing course of an effective amount of 25-hydroxyvitamin D to the patient to stabilize serum 25-hydroxyvitamin D and calcium levels followed by treatment with an agent known to increase the risk of iatrogenic hypocalcemia is effective to prevent or treat the iatrogenic hypocalcemia.

In any of the methods disclosed herein, administration of 25-hydroxyvitamin D to a patient, e.g., a patient treated with an agent that increases the risk of hypocalcemia or an anticancer agent, as described can be characterized by one or more measures described below, individually or in combination. In one aspect, the amount of 25-hydroxyvitamin D administered is effective to restore or maintain the patient's corrected serum calcium level to at least about 8.0 mg/dL, optionally in a range of about 8.3 mg/dL to about 11.6 mg/dL. In another aspect, the amount of 25-hydroxyvitamin D administered can be effective to restore or maintain the patient's corrected serum calcium level to at least about 8.3 mg/dL, 8.5 mg/dL, at least about 9.0 mg/dL, at least about 9.5 mg/dL, at least about 10 mg/dL, at least about 10.5 mg/dL, or at least about 11.0 mg/dL, optionally in a range of about 8.5 mg/dL to about 11.0 mg/dL, about 8.3 mg/dL to about 10.2 mg/dL, about 8.3 mg/dL to about 11.0 mg/dL, or about 8.5 mg/dL to about 10.2 mg/dL, for example.

In another aspect, the amount of 25-hydroxyvitamin D administered can be effective to safely increase the patient's serum level of 25-hydroxyvitamin D to at least about 30 ng/mL, optionally in a range of about 30 ng/mL to about 100 ng/mL, about 35 ng/mL to about 90 ng/mL, about 40 ng/mL to about 100 ng/mL, or about 50 ng/mL to about 100 ng/mL. In another aspect, the amount of 25-hydroxyvitamin D administered can be effective to safely increase the patient's serum level of 25-hydroxyvitamin D to at least about 35 ng/mL, at least about 40 ng/mL, at least about 50 ng/mL, at least about 60 ng/mL, at least about 70 ng/mL, at least about 80 ng/mL, at least about 90 ng/mL, at least about 100 ng/mL, at least about 150 ng/mL, at least about 200 ng/mL, at least about 250 ng/mL, or at least about 300 ng/mL.

In another aspect, the amount of 25-hydroxyvitamin D administered can be effective to decrease the patient's serum parathyroid hormone level, optionally by at least about 10%, at least about 20%, at least about 30%, at least about 40%, or at least about 50%. In another aspect, the amount of 25-hydroxyvitamin D administered can be effective to decrease the patient's serum parathyroid hormone related peptide (PTHrP) level, optionally by at least about 10%, at least about 20%, at least about 30%, at least about 40%, or at least about 50%.

In another aspect, the amount of 25-hydroxyvitamin D administered can be effective to safely increase the patient's serum level of 1,25-dihydroxyvitamin D, optionally to at least about 50 pg/mL, at least about 60 pg/mL, at least about 70 pg/mL, at least about 80 pg/mL, at least about 90 pg/mL, or at least about 100 pg/mL.

In another aspect, the amount of 25-hydroxyvitamin D administered can be effective to achieve or maintain safe serum phosphorous levels, and prevent hypophosphatemia. In another aspect, the amount of 25-hydroxyvitamin D administered can be effective to achieve or maintain serum phosphorus levels above about 2.5 mg/dL, above about 3.0 mg/dL, above about 3.5 mg/dL, above about 4.0 mg/dL, or above about 4.5 mg/dL, optionally in a range between about 2.5 mg/dL and about 4.5 mg/dL.

In another aspect, the amount of 25-hydroxyvitamin D administered can be effective to have a positive effect on the patient's serum level of a marker of bone formation compared to no treatment or treatment with an antiresorptive agent alone. For example, the amount of 25-hydroxyvitamin D administered can be effective to increase the patient's serum level of a marker of bone formation, e.g., bone morphogenetic protein or osteocalcin, by at least about 10%, at least about 20%, at least about 30%, at least about 40%, or at least about 50%, compared to no treatment or treatment with an antiresorptive agent alone. In another aspect, the amount of 25-hydroxyvitamin D administered can be effective to decrease the patient's serum level of a marker of bone resorption, optionally by at least 10%, at least 20%, at least about 30%, at least about 40%, or at least about 50%, compared to no treatment levels or treatment with an antiresorptive agent alone. In another aspect, the amount of 25-hydroxyvitamin D administered can effective to mitigate the increase in the patient's serum level of a marker of bone resorption compared to no treatment or treatment with an antiresorptive agent alone. In various embodiments, the marker of bone resorption is selected from the group consisting of PTHrP, FGF23, NTX, CTX, TRAC-5b, and combinations thereof.

In another aspect, the amount of 25-hydroxyvitamin D administered can be effective to decrease or increase the patient's serum level of an immune meditating cytokine, e.g. C-reactive protein (CRP), interleukin 12, or interleukin 10, optionally by at least about 10%, at least about 20%, at least about 30%, at least 40%, or at least about 50%. In another aspect, the amount of 25-hydroxyvitamin D can be effective to increase the spot calcium/creatinine (Ca/Cr) ratio.

In another aspect, the amount of 25-hydroxyvitamin D administered can be effective to maintain or decrease the patient's tumor burden. Tumor burden may be measured using assays known in the art, e.g., radiography, computed tomography (CT), or magnetic resonance imaging (MRI). Tumor burden may also be assessed by measuring one or more markers of tumor burden. In another aspect, the amount of 25-hydroxyvitamin D administered can be effective to decrease the patient's serum level of a marker of tumor burden, optionally by at least about 10%, at least about 20%, at least about 30%, at least about 40%, or at least about 50%, compared to no treatment or treatment with an anticancer agent and/or an agent that increases the risk of hypocalcemia alone. In another aspect, the amount of 25-hydroxyvitamin D administered can be effective to mitigate the increase in the patient's tumor burden or serum level of a marker of tumor burden, compared to no treatment or treatment with an anticancer agent and/or agent that increases the risk of hypocalcemia. In embodiments, the marker of tumor burden can be optionally selected from the group consisting of CEA, CA 125, CA15-3, CA 27-29, prostate specific antigen (PSA), and combinations thereof.

In one class of embodiments, the effective amount of 25-hydroxyvitamin D is co-administered with an agent that increases the risk of hypocalcemia and/or an anticancer agent. The present disclosure also provides a kit comprising (a) 25-hydroxyvitamin D, (b) an agent that increases the risk of hypocalcemia and/or an anticancer agent, and (c) instructions for co-administering effective amounts of (a) and (b) to a patient in need thereof. The indications and usage of the agent(s) co-administered with 25-hydroxyvitamin D according to the present methods are not particularly limited, and can be equivalent to those already taught in the literature.

The methods of the present disclosure are suitable for treating patients having a condition responsive to administration of 25-hydroxyvitamin D as described. In one type of embodiment, the patient that has osteoporosis. In another type of embodiment, the patient that has hungry bone syndrome. In another type of embodiment, the patient has impaired renal function, e.g., a patient having Chronic Kidney Disease Stage 1, 2, 3, 4, or 5.

In another type of embodiment, the patient has cancer, optionally a cancer selected from the group consisting of bone cancer, bladder cancer, breast cancer, colon cancer, endometrial cancer, kidney cancer, leukemia, lung cancer, lymphoma, pancreatic cancer, prostate cancer, skin cancer, thyroid cancer, and metastatic forms thereof. In one embodiment, the patient has cancer and a bone tumor, i.e., a bone metastasis from a solid tumor. For example, the patient may have metastatic bone cancer, metastatic prostate cancer, metastatic lung cancer, and/or metastatic breast cancer.

Optionally, the patient has cancer and is receiving, has previously received, or will receive, treatment with an anticancer agent. Exemplary classes of anticancer agents include, but are not limited to, an aromatase inhibitor; an anti-estrogen; an anti-androgen; a gonadorelin agonist; a topoisomerase I inhibitor; a topoisomerase II inhibitor; a microtubule active agent; an alkylating agent; a retinoid, a carotenoid, or a tocopherol; a cyclooxygenase inhibitor; an MMP inhibitor; a mTOR inhibitor; an antimetabolite; a platin compound; a methionine aminopeptidase inhibitor; a bisphosphonate; an antiproliferative antibody; a heparanase inhibitor; an inhibitor of Ras oncogenic isoforms; a telomerase inhibitor; a proteasome inhibitor; a Flt-3 inhibitor; an Hsp90 inhibitor; a kinesin spindle protein inhibitor; a MEK inhibitor; an antitumor antibiotic; a nitrosourea, a compound targeting/decreasing protein or lipid kinase activity, a compound targeting/decreasing protein or lipid phosphatase activity, an antiangiogenic compound, and combinations thereof. In various types of embodiments, the patient can be treated with an anticancer agent selected from the group consisting of azacitidine, axathioprine, bevacizumab, bleomycin, capecitabine, carboplatin, chlorabucil, cisplatin, cyclophosphamide, cytarabine, daunorubicin, docetaxel, doxifluridine, doxorubicin, epirubicin, etoposide, fluorouracil, gemcitabine, herceptin, idarubicin, mechlorethamine, melphalan, mercaptopurine, methotrexate, mitoxantrone, oxaliplatin, paclitaxel, tafluposide, teniposide, tioguanine, retinoic acid, valrubicin, vinblastine, vincristine, vindesine, vinorelbine, receptor tyrosine kinase inhibitors, and combinations thereof.

Optionally, the patient having a condition described above to be treated with 25-hydroxyvitamin D is receiving, has previously received, or will receive, treatment with an agent that increases the risk of hypocalcemia, optionally an agent selected from the group consisting of an antiresorptive agent, an anticonvulsant agent, a corticosteroid, an antihypercalcemia agent, an antimicrobial agent, and combinations thereof. In one type of embodiment, the agent that increases the risk of hypocalcemia is an antihypercalcemia agent, optionally the antihypercalcemia agent cinacalcet. In another type of embodiment, the agent that increases the risk of hypocalcemia is an antiresorptive agent, optionally selected from the group consisting of bisphosphonates, selective estrogen receptor modulators, calcitonin, hormones, and monoclonal antibodies. In one type of embodiment, the antiresorptive agent comprises a RANKL inhibitor, optionally the RANKL inhibitor denosumab. In another type of embodiment, the antiresorptive agent comprises a bisphosphonate, optionally the bisphosphonate zoledronic acid. Optionally, a patient having cancer is receiving, has previously received, or will receive, treatment with an agent that increases the risk of hypocalcemia and an anticancer agent.

For each of the foregoing measures, it is contemplated that adjunctive therapy with 25-hydroxyvitamin D will achieve such increases, decreases, and/or delays to a greater degree compared to administering the agent that increases the risk of hypocalcemia and/or anticancer agent alone. In another aspect, it is contemplated that the adjunctive therapy with 25-hydroxyvitamin D will achieve such increases, decreases, and/or delays to a greater degree compared to co-administering the agent that increases the risk of hypocalcemia with cholecalciferol, optionally with an anticancer agent. It is contemplated that the adjunctive therapy with 25-hydroxyvitamin D will achieve such increases, decreases, and/or delays to a greater degree compared to co-administering the agent that increases the risk of hypocalcemia with ergocalciferol, optionally with an anticancer agent. It is also contemplated that adjunctive therapy with 25-hydroxyvitamin D will mitigate, i.e., lessen the severity of, undesirable effect compared to administering the agent that increases the risk of hypocalcemia and/or anticancer agent alone or the antiresorptive agent with cholecalciferol or ergocalciferol, optionally with an anticancer agent. Examples of undesired effects include, but are not limited to, an increase or decrease of serum calcium or phosphorous to a level outside the normal range, a decrease in blood levels of a bone formation marker, an increase in blood levels of a bone resorption marker, and an increase in tumor burden (e.g., an increase in a marker of tumor progression).

The present disclosure also contemplates compositions comprising oral or intravenous formulations of 25-hydroxyvitamin D and related methods of administration. Such compositions and related methods of administration can be selected to have one or more features including increasing blood levels of 25-hydroxyvitamin D without the potential first-pass effects of 25-hydroxyvitamin D prohormones in the duodenum; without supraphysiological surges in intralumenal, intracellular and blood levels of 25-hydroxyvitamin D and their consequences; without causing substantially increased catabolism of the administered 25-hydroxyvitamin D; and without causing serious side effects associated with Vitamin D supplementation, namely Vitamin D toxicity.

In one type of embodiment, modified release compositions intended for oral administration in accordance with the present invention are designed to contain a concentration of 25-hydroxyvitamin D (e.g. 25-hydroxyvitamin D₃, or a combination of 25-hydroxyvitamin D₂ and 25-hydroxyvitamin D₃) of 1 to 1000 mcg per unit dose, or 1 to 500 mcg per unit dose or 1 to 100 mcg per dose, or 1 to 50 mcg per dose, or 10 to 40 mcg per dose, for example 30 mcg per dose or 60 mcg per dose, or 90 mcg per dose, and are prepared in such a manner as to effect controlled or substantially constant release of the 25-hydroxyvitamin D into the gastrointestinal tract of a subject over an extended period of time. In one embodiment, the 25-hydroxyvitamin D is 25-hydroxyvitamin D₃. In another embodiment, the 25-hydroxyvitamin D is a combination of 25-hydroxyvitamin D₃ and 25-hydroxy vitamin D₂ and are useful in supporting both the Vitamin D₃ and Vitamin D₂ endocrine systems. Currently available oral Vitamin D supplements and the previously marketed oral formulation of 25-hydroxyvitamin D₃ have supported just one or the other system. In one type of embodiment, the release can be in the ileum or later, for example in the colon. In another type embodiment, the composition can result in a substantially increased absorption of 25-hydroxyvitamin D via transport on DBP and decreased absorption via transport in chylomicrons. In another type of embodiment, the composition can result in maintenance of substantially constant blood levels of 25-hydroxyvitamin D during the 24-hour post-dosing period. Examples of modified release compositions of 25-hydroxyvitamin D are described in U.S. Pat. Nos. 8,207,149, 8,361,488, and 8,426,391, and 14,213,285, incorporated herein by reference.

A composition of the present disclosure comprising 25-hydroxyvitamin D optionally further comprises an agent that increases the risk of hypocalcemia or an anticancer agent.

In one type of embodiment, the 25-hydroxyvitamin D is administered orally. For example, the 25-hydroxyvitamin D can be administered in an oral modified release formulation. In the alternative, the 25-hydroxyvitamin D can be administered in an oral immediate release formulation in multiple daily doses in order to produce a pharmacokinetic profile of serum 25-hydroxyvitamin D that is similar to that achieved by an oral modified or sustained release formulation.

The preparation of a modified release form of 25-hydroxyvitamin D suitable for oral administration can be carried out according to many different techniques. For example, one or more 25-hydroxyvitamin D compounds can be dispersed within a matrix, i.e., a unique mixture of rate controlling constituents and excipients in carefully selected ratios within the matrix, and optionally encased with a coating material. In another alternative, various coating techniques can be utilized to control the rate and/or the site of the release of the 25-hydroxyvitamin D from the pharmaceutical formulation. For example, the dissolution of the coating may be triggered by the pH of the surrounding media, and the resulting gradual dissolution of the coating over time exposes the matrix to the fluid of the local environment. In one type of embodiment, after the coating becomes permeable, 25-hydroxyvitamin D diffuses from the outer surface of the matrix. When this surface becomes exhausted or depleted of 25-hydroxyvitamin D, the underlying stores begin to be depleted by diffusion through the disintegrating matrix to the external solution. In another type of embodiment, release of 25-hydroxyvitamin D is by gradual disintegration or erosion of the matrix, e.g., via solubility of one or more components of the matrix and/or by lack of physical integrity.

In one aspect, a formulation in accordance with the present invention provides one or more 25-hydroxyvitamin D compounds within a matrix that releasably binds the ingredients for sustained release, e.g., when exposed to the contents of the ileum and/or colon.

Optionally, the 25-hydroxyvitamin D-containing matrix can be suitably covered with a coating that is resistant to disintegration in gastric juices. The coated modified release formulation of 25-hydroxyvitamin D is then administered orally to subjects, e.g., animals or human patients. As the formulation travels through the proximal portion of the small intestine, the enteric coating becomes progressively more permeable but, in a suitable embodiment, it provides a persisting structural framework around the 25-hydroxyvitamin D-containing matrix. The 25-hydroxyvitamin D-containing matrix becomes significantly exposed to intestinal fluids in the ileum through the permeable overcoating, and the 25-hydroxyvitamin D is then gradually released by simple diffusion and/or slow disintegration of the matrix.

Once released into the lumen of the ileum, the 25-hydroxyvitamin D is absorbed into the lymphatic system or into the portal bloodstream, where it is bound to and transported by the DBP. In this embodiment, the 25-hydroxyvitamin D is primarily absorbed at a point beyond the duodenum and jejunum. These proximal portions of the small intestine can respond to high intralumenal levels of 25-hydroxyvitamin D and in the process, can catabolize significant quantities of the 25-hydroxyvitamin D. By substantially delaying 25-hydroxyvitamin D release until the ileum and/or colon, the pharmaceutical composition described herein virtually eliminates these potential first-pass effects in the proximal intestine and reduces unwanted catabolism. Significant catabolism of administered 25-hydroxyvitamin D prior to absorption into the bloodstream significantly lowers its bioavailability. Elimination of first-pass effects reduces the risk of Vitamin D toxicity. Substantially delayed release of 25-hydroxyvitamin D (i.e., beyond the duodenum and jejunum) markedly decreases the amount of 25-hydroxyvitamin D that is incorporated and absorbed from the small intestine via chylomicrons (since chylomicron formation and absorption occurs primarily in the jejunum) and correspondingly increases the amount of 25-hydroxyvitamin D that is absorbed directly through the intestinal wall and onto DBP circulating in lymph or portal blood.

In one embodiment of the invention, a controlled release oral formulation of 25-hydroxyvitamin D is prepared generally according to the following procedure. A sufficient quantity of 25-hydroxyvitamin D is completely dissolved in a minimal volume of USP-grade absolute ethanol (or other suitable solvent) and mixed with appropriate amounts and types of pharmaceutical-grade excipients to form a matrix which is solid or semi-solid at both room temperature and at the normal temperature of the human body. The matrix is completely or almost entirely resistant to digestion in the stomach and upper small intestine, and it gradually disintegrates in the lower small intestine and/or colon.

In a suitable formulation, the matrix binds the 25-hydroxyvitamin D compound(s) and permits a slow, relatively steady, e.g. substantially constant, release of 25-hydroxyvitamin D over a period of four to eight hours or more, by simple diffusion and/or gradual disintegration, into the contents of the lumen of the lower small intestine and/or colon. The formulation optionally further has an enteric coating that partially dissolves in aqueous solutions having a pH of about 7.0 to 8.0, or simply dissolves slowly enough that significant release of 25-hydroxyvitamin D is delayed until after the formulation passes through the duodenum and jejunum.

As discussed above, the means for providing the controlled release of 25-hydroxyvitamin D may be selected from any suitable controlled release delivery system, including any of the known controlled release delivery systems of an active ingredient over a course of about four or more hours, including the wax matrix system, and the EUDRAGIT RS/RL system (Rohm Pharma, GmbH, Weiterstadt, Germany).

The wax matrix system provides a lipophilic matrix. The wax matrix system may utilize, for example, beeswax, white wax, cachalot wax or similar compositions. The active ingredient(s) are dispersed in the wax binder which slowly disintegrates in intestinal fluids to gradually release the active ingredient(s). The wax binder that is impregnated with 25-hydroxyvitamin D can be loaded into softgel capsules. A softgel capsule may comprise one or more gel-forming agents, e.g., gelatin, starch, carrageenan, and/or other pharmaceutically acceptable polymers. In one embodiment, partially crosslinked soft gelatin capsules are used. As another option, vegetable-based capsules can be used. The wax matrix system disperses the active ingredient(s) in a wax binder which softens at body temperature and slowly disintegrates in intestinal fluids to gradually release the active ingredient(s). The system suitably can include a mixture of waxes, with the optional addition of oils, to achieve a melting point which is higher than body temperature, but lower than the melting temperature of the selected formulations used to create the shell of a soft or hard capsule, or vegetable capsule shell, or other formulation used to create a shell casing or other coating.

Specifically, in one suitable embodiment, the waxes selected for the matrix are melted and thoroughly mixed. The desired quantity of oils are subsequently added, followed by sufficient mixing for homogenization. The waxy mixture is then gradually cooled to a temperature just above its melting point. The desired amount of 25-hydroxyvitamin D, dissolved in ethanol, is uniformly distributed into the molten matrix, and the matrix is loaded into capsules, for example vegetable-based or gelatin-based capsules. The filled capsules optionally are treated for appropriate periods of time with a solution containing an aldehyde, such as acetaldehyde, to partially crosslink a polymer, e.g., gelatin, in the capsule shell, when used. The capsule shell becomes increasingly crosslinked, over a period of several weeks and, thereby, more resistant to dissolution in the contents of stomach and upper intestine. When properly constructed, this gelatin shell will gradually dissolve after oral administration and become sufficiently porous (without fully disintegrating) by the time it reaches the ileum to allow the 25-hydroxyvitamin D to diffuse slowly from the wax matrix into the contents of the lower small intestine and/or colon.

Examples of other lipid matrices suitable for use with the methods of the invention include one or more of glycerides, fatty acids and alcohols, and fatty acid esters.

In one embodiment, a formulation may comprise an oily vehicle for the 25-hydroxyvitamin D compound. Any pharmaceutically-acceptable oil can be used. Examples include animal (e.g., fish), vegetable (e.g., soybean), and mineral oils. The oil preferably will readily dissolve the 25-hydroxyvitamin D compound used. Oily vehicles can include non-digestible oils, such as mineral oils, particularly liquid paraffins, and squalene. The ratio between the wax matrix and the oily vehicle can be optimized in order to achieve the desired rate of release of the 25-hydroxyvitamin D compound. Thus, if a heavier oil component is used, relatively less of the wax matrix can be used, and if a lighter oil component is used, then relatively more wax matrix can be used. In one embodiment, the particular choice of oily vehicle provides a controlled release so that absorption of 25-hydroxyvitamin D is delayed until the formulation reaches the ileum and/or colon.

Another suitable controlled-release oral drug delivery system is the EUDRAGIT RL/RS system in which the active 25-hydroxyvitamin D ingredient is formed into granules having a dimension of 25/30 mesh. The granules are then uniformly coated with a thin polymeric lacquer, which is water-insoluble but slowly water-permeable. The coated granules can be mixed with optional additives including one or more of antioxidants, stabilizers, binders, lubricants, processing aids and the like. The mixture may be compacted into a tablet which, prior to use, is hard and dry and can be further coated, or it may be poured into a capsule. After the tablet or capsule is swallowed and comes into contact with the aqueous intestinal fluids, the thin lacquer begins to swell and slowly allows permeation by intestinal fluids. As the intestinal fluid slowly permeates the lacquer coating, the contained 25-hydroxyvitamin D is slowly released. By the time the tablet or capsule has passed through the small intestine, about four to eight hours or more later, the 25-hydroxyvitamin D will have been slowly, but completely, released. Accordingly, the ingested tablet will release a stream of 25-hydroxyvitamin D, as well as any other active ingredient.

The EUDRAGIT system is comprised of high permeability lacquers (RL) and low permeability lacquers (RS). RS is a water-insoluble film former based on neutral swellable methacrylic acids esters with a small proportion of trimethylammonioethyl methacrylate chlorides; the molar ratio of the quaternary ammonium groups to the neutral ester group is about 1:40. RL is also a water insoluble swellable film former based on neutral methacrylic acid esters with a small portion of trimethylammonioethyl methacrylate chloride, the molar ratio of quaternary ammonium groups to neutral ester groups is about 1:20. The permeability of the coating and thus the time course of drug release can be titrated by varying the proportion of RS to RL coating material. For further details of the Eudragit RL/RS system, reference is made to technical publications available from Rohm Tech, Inc. 195 Canal Street, Maiden, Mass., 02146 and K. Lehmann, D. Dreher “Coating of tablets and small particles with acrylic resins by fluid bed technology,” Int. J. Pharm. Tech. & Prod. Mfr. 2(r), 31-43 (1981), incorporated herein by reference.

Other examples of insoluble polymers include polyvinyl esters, polyvinyl acetals, polyacrylic acid esters, butadiene styrene copolymers and the like.

In one embodiment, once the coated granules are either formed into a tablet or put into a capsule, the tablet or capsule is coated with an enteric-coating material which dissolves at a pH of 7.0 to 8.0. One such pH-dependent enteric-coating material is EUDRAGIT L/S which dissolves in intestinal fluid, but not in the gastric juices. Other enteric-coating materials may be used such as cellulose acetate phthalate (CAP), which is resistant to dissolution by gastric juices, but readily disintegrates due to the hydrolytic effect of the intestinal esterases.

In one embodiment, the particular choice of enteric-coating material and controlled release coating material provides a controlled and substantially constant release over a period of 4 to 8 hours or more so that substantial release is delayed until the formulation reaches the ileum. Optionally, a controlled release composition in accordance with the present disclosure, when administered once a day, can suitably provide substantially constant intralumenal, intracellular and blood 25-hydroxyvitamin D levels compared to an equal dose of an immediate release composition of 25-hydroxyvitamin D administered once a day.

The dosage forms may also contain adjuvants, such as preserving or stabilizing adjuvants. For example, a preferred formulation includes 25-hydroxyvitamin D (e.g., about 30 mcg, about 60 mcg, or about 90 mcg 25-hydroxyvitamin D₃), about 2 wt % anhydrous ethanol, about 10 wt % lauroyl polyoxylglycerides, about 20 wt % hard paraffin, about 23 wt % glycerol monostearate, about 35 wt % liquid paraffin or mineral oil, about 10 wt % hydroxypropyl methylcellulose, and optionally a small amount of preservative (e.g., butylated hydroxytoluene). Formulations according to the invention may also contain other therapeutically valuable substances or may contain more than one of the compounds specified herein and in the claims in admixture.

As an alternative to oral 25-hydroxyvitamin D, intravenous administration of 25-hydroxyvitamin D is also contemplated. In one embodiment, the 25-hydroxyvitamin D is administered as an sterile intravenous bolus, optionally a bolus injection of a composition that results in a sustained release profile. In another embodiment, the 25-hydroxyvitamin D is administered via gradual injection/infusion, e.g., over a period of 1 to 5 hours, to effect controlled or substantially constant release of the 25-hydroxyvitamin D directly to DBP in the blood of the patient. For example, the composition may be injected or infused over a course of at least about 1 hour, at least about 2 hours, at least about 3 hours, at least about 4 hours, at least about 5 hours, or at least about 6 hours. In one embodiment, the composition intended for intravenous administration in accordance with the present invention is designed to contain a concentration of the 25-hydroxyvitamin D compound(s) of 1 to 100 mcg per unit dose. Sterile, isotonic formulations of 25-hydroxyvitamin D may be prepared by dissolving 25-hydroxyvitamin D in absolute ethanol, propylene glycol or another suitable solvent, and combining the resulting solution with one or more surfactants, salts and preservatives in appropriate volumes of water for injection. Such formulations can be administered slowly from syringes, for example, via heparin locks, or by addition to larger volumes of sterile solutions (e.g., saline solution) being steadily infused over time. In one embodiment, the composition can be co-injected or co-infused with an anticancer agent.

In another aspect, administration of an effective amount of a composition of the present disclosure can be effective to safely achieve supraphysiologic levels of 25-hydroxyvitamin D and/or 1,25-dihydroxyvitamin D i.e., without causing hypercalcemia and/or hyperphosphatemia.

Advantageously, adjunctive therapy comprising 25-hydroxyvitamin D and an agent that increases the risk of hypocalcemia and/or an anticancer agent, optionally together with other therapeutic agents, can be orally or intravenously administered in accordance with the above described embodiments in dosage amounts of from 1 to 100 mcg per day, with the preferred dosage amounts of from 5 to 50 mcg per day. If the 25-hydroxyvitamin D and an agent that increases the risk of hypocalcemia and/or an anticancer agent are co-administered in combination with other therapeutic agents, the proportions of each of the compounds in the combination being administered will be dependent on the particular disease state being addressed. For example, one may choose to orally administer 25-hydroxyvitamin D with one or more calcium salts (intended as a calcium supplement or dietary phosphate binder), calcimimetics, nicotinic acid, iron, phosphate binders, cholecalciferol, ergocalciferol, active Vitamin D sterols, or glycemic and hypertension control agents. In addition, one may choose to intravenously administer 25-hydroxyvitamin D with cholecalciferol, ergocalciferol, active Vitamin D sterols, or glycemic and hypertension control agents. In practice, higher doses of the compounds of the present disclosure are used where therapeutic treatment of a disease state is the desired end, while the lower doses are generally used for prophylactic purposes, it being understood that the specific dosage administered in any given case will be adjusted in accordance with the specific compounds being administered, the disease to be treated, the condition of the subject and the other relevant medical facts that may modify the activity of the drug or the response of the subject, as is well known by those skilled in the art.

The present invention is further explained by the following examples which should not be construed by way of limiting the scope of the present invention.

### Example 1

One Embodiment of a Modified Release Formulation for Oral Administration

Purified yellow beeswax and fractionated coconut oil are combined in a ratio of 1:1 and heated with continuous mixing to 75 degrees Celsius until a uniform mixture is obtained. The wax mixture is continuously homogenized while cooled to approximately 45 degrees Celsius. The active compounds, 25-hydroxyvitamin D₂ and 25-hydroxyvitamin D₃, in a ratio of 1:1, are dissolved in absolute ethanol and the ethanolic solution is added, with continuous homogenization, to the molten wax mixture. The amount of ethanol added is in the range of 1 to 2 v/v %. Mixing is continued until the mixture is uniform. The uniform mixture is loaded into soft gelatin capsules. The capsules are immediately rinsed to remove any processing lubricant(s) and briefly immersed in an aqueous solution of acetaldehyde in order to crosslink the gelatin shell. The concentration of the acetaldehyde solution and the immersion time is selected to achieve crosslinking to the desired degree, as determined by near-infrared spectrophotometry. The finished capsules are washed, dried and packaged.

### Example 2

One Embodiment of a Formulation for Intravenous Administration

TWEEN Polysorbate 20 is warmed to approximately 50 to 60 degrees Fahrenheit, and 25-hydroxyvitamin D₃, dissolved in a minimal volume of absolute ethanol, is added with continuous stirring. The resulting uniform solution of 25-hydroxyvitamin D₃, absolute ethanol and TWEEN Polysorbate 20 is transferred to a suitable volume of water for injection, which has been thoroughly sparged with nitrogen to remove all dissolved oxygen. Sodium chloride, sodium ascorbate, sodium phosphate (dibasic and monobasic), and disodium edetate are added, followed by sufficient stirring under a protective nitrogen atmosphere, to produce an isotonic homogeneous mixture containing, per 2 mL unit volume: 20 mcg of 25-hydroxyvitamin D₃; less than 0.01% absolute ethanol; 0.40% (w/v) TWEEN Polysorbate 20; 0.15% (w/v) sodium chloride; 1.00% (w/v) sodium ascorbate; 0.75% (w/v) sodium phosphate dibasic anhydrous; 0.18% (w/v) sodium phosphate monobasic monohydrate; and, 0.11% (w/v) disodium edetate. The mixture is sterilized by filtration and filled, with suitable protection from oxygen contamination, into amber glass ampules having an oxygen headspace of less than 1%.

### Example 3

Pharmacokinetics Testing in Dogs

Twenty male beagle dogs are divided randomly into two comparable groups and receive no supplemental Vitamin D for the next 30 days. At the end of this time, each dog in Group #1 receives a single softgel capsule containing 25 mcg of 25-hydroxyvitamin D₂ prepared in a controlled release formulation similar to the one disclosed in Example 1. Each dog in the other group (Group #2) receives a single immediate-release softgel capsule containing 25 mcg of 25-hydroxyvitamin D₂ dissolved in medium chain triglyceride oil. All dogs have received no food for at least 8 hours prior to dosing. Blood is drawn from each dog at 0, 0.5, 1, 1.5, 2, 3, 4, 6, 9, 15, 24, 36, and 72 hours after dose administration. The collected blood is analyzed for the contained levels of 25-hydroxyvitamin D, and the data are analyzed by treatment group. Dogs in Group #1 show a slower rise and a lower maximum (Cₘₐₓ) in mean blood levels of 25-hydroxyvitamin D than dogs in Group #2. However, dogs in Group #1 show a more prolonged elevation of mean blood levels of 25-hydroxyvitamin D₂ relative to dogs in Group #2, despite the fact that the Cₘₐₓ recorded in Group #1 is lower. The mean area under the curve (AUC), corrected for predose background levels (recorded at t=0), is substantially greater for Group #1 for 25-hydroxyvitamin D. These procedures demonstrate that administration of 25-hydroxyvitamin D₂ in the formulation described in this invention to dogs results in blood levels of 25-hydroxyvitamin D which rise much more gradually and remain more stable than after dosing with the same amount of 25-hydroxyvitamin D₂ formulated for immediate release (in medium chain triglyceride oil). The greater AUC calculated for blood levels of 25-hydroxyvitamin D in Group #1 demonstrates that the bioavailability of 25-hydroxyvitamin D₂ formulated as described herein is markedly improved.

### Example 4

Pharmacokinetics Testing in Healthy Normal Volunteers

Sixteen healthy non-obese adults, aged 18 to 24 years, participate in an 11-week pharmacokinetic study in which they receive successively, and in a double-blinded fashion, two formulations of 25-hydroxyvitamin D₂. One of the formulations (Formulation #1) is a softgel capsule containing 100 mcg of 25-hydroxyvitamin D₂ prepared in a controlled release formulation similar to the one disclosed in Example 1. The other formulation (Formulation #2) is an immediate-release softgel capsule of identical appearance containing 100 mcg of 25-hydroxyvitamin D₂ dissolved in medium chain triglyceride oil. For 60 days prior to study start and continuing through study termination, the subjects abstain from taking other Vitamin D supplements. On Days 1, 3 and 5 of the study, all subjects provide fasting morning blood samples to establish pre-treatment baseline values. On the morning of Day 8, the subjects provide an additional fasting blood sample (t=0), and are randomly assigned to one of two treatment groups. Both groups are dosed with a single test capsule prior to eating breakfast: One group receives a capsule of Formulation #1 and the other group receives a capsule of Formulation #2. Blood is drawn from each subject at 0.5, 1, 1.5, 2, 3, 4, 6, 8, 10, 12, 15, 24, 36, 48, 72 and 108 hours after dose administration. On the morning of Day 70, the subjects provide additional fasting morning blood samples (t=0) and are dosed with a single capsule of the other test formulation prior to eating breakfast. Blood is again drawn from each subject at 0.5, 1, 1.5, 2, 3, 4, 6, 8, 10, 12, 15, 24, 36, 48, 72 and 108 hours after dose administration. All collected blood is analyzed for the contained levels of 25-hydroxyvitamin D, and the data are analyzed by treatment formulation after correction for baseline content. Formulation #1 is found to produce a slower rise and a lower Cₘₐₓ in mean blood levels of 25-hydroxyvitamin D than Formulation #2. However, Formulation #1 also produces a more prolonged elevation of mean blood levels of 25-hydroxyvitamin D₂ relative to Formulation #2, despite the fact that the recorded Cₘₐₓ is lower. The mean AUC for 25-hydroxyvitamin D₂ is substantially greater after administration of Formulation #1. These procedures demonstrate that administration of 25-hydroxyvitamin D₂ in the formulation described in this invention to healthy human adults results in blood levels of 25-hydroxyvitamin D₂ which rise much more gradually and remain more stable than after dosing with the same amount of 25-hydroxyvitamin D₂ formulated for immediate release (in medium chain triglyceride oil). The greater AUC calculated for blood levels of 25-hydroxyvitamin D₂ after dosing with Formulation #1 demonstrates that the bioavailability of 25-hydroxyvitamin D₂ formulated as described herein is better.

### Example 5

Efficacy Study in Healthy Adult Male Volunteers with Vitamin D Insufficiency

The effectiveness of three different formulations of Vitamin D in restoring serum total 25-hydroxyvitamin D to optimal levels (>30 ng/mL) is examined in a 23-day study of healthy non-obese men diagnosed with Vitamin D insufficiency. One of the formulations (Formulation #1) is a sustained release softgel capsule containing 30 mcg of 25-hydroxyvitamin D₃ prepared as illustrated in this disclosure. The second formulation (Formulation #2) is an immediate-release softgel capsule of identical appearance containing 50,000 IU of ergocalciferol dissolved in medium chain triglyceride oil. The third formulation (Formulation #3) is an immediate-release softgel capsule, also of identical appearance, containing 50,000 IU of cholecalciferol dissolved in medium chain triglyceride oil. A total of 100 healthy Caucasian and African-American men participate in this study, all of whom are aged 30 to 45 years and have serum 25-hydroxyvitamin D levels between 15 and 29 ng/mL (inclusive). All subjects abstain from taking other Vitamin D supplements for 60 days before study start and continuing through study termination, and from significant sun exposure. On Day 1 and 2 of the study, all subjects provide fasting morning blood samples to establish pre-treatment baseline values of serum total 25-hydroxyvitamin D. On the morning of Day 3, the subjects provide an additional fasting blood sample (t=0), are randomly assigned to one of four treatment groups, and are dosed with a single test capsule prior to eating breakfast: The subjects in Group #1 each receive a single capsule of Formulation #1, and the subjects in Groups #2 and #3 each receive a single capsule of Formulation #2 or Formulation #3, respectively. Subjects in Group #4 receive a matching placebo capsule. Subjects in Group #1 each receive an additional capsule of Formulation #1 on the mornings of Days 4 through 22 before breakfast, but subjects in Groups #2, #3 and #4 receive no additional capsules. A fasting morning blood sample is drawn from each subject, irrespective of treatment group, on Days 4, 5, 6, 10, 17 and 23 (or 1, 2, 3, 7, 14 and 20 days after the start of dosing). All collected blood is analyzed for the contained levels of 25-hydroxyvitamin D, and the data are analyzed by treatment group after correction for baseline values. Subjects in all four treatment groups exhibit mean baseline serum 25-hydroxyvitamin D levels of approximately 16 to 18 ng/mL, based on analysis of fasting blood samples drawn on Days 1 through 3. Subjects in Group #4 (control group) show no significant changes in mean serum total 25-hydroxyvitamin D over the course of the study. Subjects in Group #1 show a steadily increasing mean serum total 25-hydroxyvitamin D reaching at least 30 ng/mL by Day 23. In marked contrast, subjects in Group #2 exhibit marked increases in mean serum 25-hydroxyvitamin D for the first few days post-dosing, reaching a maximum of just above 25 ng/mL, and then rapidly declining thereafter. By study end, serum total 25-hydroxyvitamin D is significantly lower than baseline in Group #2. Subjects in Group #3 exhibit continuing increases in mean serum total 25-hydroxyvitamin D through the first 2 weeks after dosing with gradual, but progressive, decreases occurring thereafter. By study end, mean serum total 25-hydroxyvitamin D is below 30 ng/mL. The data from this study demonstrate that administration of 600 mcg of 25-hydroxyvitamin D₃, formulated as described herein and administered at a dose of 30 mcg per day for 20 days, is substantially more effective in restoring low serum levels of 25-hydroxyvitamin D to optimal levels than immediate-release formulations of 50,000 IU of either ergocalciferol or cholecalciferol administered in single doses, as currently recommended by the NKF and other leading experts on oral Vitamin D replacement therapy.

### Example 6

Efficacy Study in Osteoporosis Patients Treated with an Antiresorptive Agent

The effectiveness of oral modified release 25-hydroxyvitamin D₃ in restoring serum total 25-hydroxyvitamin D to optimal levels (>30 ng/mL), and thereby optimizing the effectiveness of an antiresorptive agent at increasing bone mineral density, is examined in a 24-month study of adult male and female patients with osteoporosis. In a randomized, double-blind controlled study, patients are treated with denosumab (60 mg at the start of treatment and again every six months). All denosumab-treated patients are randomized to receive daily oral treatment with one softgel capsule containing either 30 mcg of 25-hydroxyvitamin D₃ in a modified release formulation or 400 IU of Vitamin D₃ (cholecalciferol) in an immediate release formulation. A total of 500 subjects participate in this study, 250 male and 250 female, all of whom are aged 60 to 85 years (inclusive), have bone mineral density T-scores between −2.0 and −4.0, and have serum total 25-hydroxyvitamin D levels less than 30 ng/mL at the time of enrollment. All subjects receive calcium supplements (500 mg/day) and abstain from taking other Vitamin D supplements for 60 days before study start and continuing through study termination, and from significant sun exposure. All subjects begin daily dosing with softgel capsules at the start of denosumab treatment. Serum total 25-hydroxyvitamin D, PTH, calcium, phosphorus, N- and C-telopeptides, and P1NP, and urinary calcium, phosphorus and creatinine, are measured monthly. Bone mineral density at four sites (total hip, femoral neck, ⅓ radius and lumbar spine) is determined at quarterly intervals.

After 3 months, the daily softgel capsule dosage is maintained unchanged in patients whose serum total 25-hydroxyvitamin D is between 50 and 90 ng/mL, and increased by one capsule in patients whose serum total 25-hydroxyvitamin D is below 50 ng/mL. The dosage is immediately lowered by one capsule per day in patients whose serum total 25-hydroxyvitamin D rises above 100 ng/mL or whose serum calcium is confirmed above 10.3 mg/dL. After 6 to 9 months, all subjects exhibit serum total 25-hydroxyvitamin D levels that remain essentially stable with continuing dosing and rise to approximately 50 to 100 ng/mL with 25-hydroxyvitamin D₃ treatment or to approximately 25 to 35 ng/mL with Vitamin D₃ treatment. In patients treated with 25-hydroxyvitamin D₃, the incidence of hypocalcemia and severity of secondary hyperparathyroidism is markedly reduced once stable dosing has been achieved. However, in patients treated with Vitamin D₃, hypocalcemia and secondary hyperparathyroidism occur more frequently. After 24 months of treatment, the patients treated with denosumab and 25-hydroxyvitamin D₃ are found to have higher and more consistent serum levels of 25-hydroxyvitamin D₃ and lower serum PTH levels than patients treated with denosumab and Vitamin D₃. Patients treated with denosumab and 25-hydroxyvitamin D₃ are also found to have larger increases in bone mineral density than patients treated with denosumab and Vitamin D₃. Data from this study demonstrate that the modified release formulation of 25-hydroxyvitamin D₃ is effective at increasing serum total 25-hydroxyvitamin D without causing unacceptable side effects related to calcium and PTH metabolism and at augmenting the increases in bone mineral density produced by denosumab.

### Example 7

Efficacy Study in Prostate Cancer Patients

The effectiveness of oral modified release 25-hydroxyvitamin D₃ in restoring serum total 25-hydroxyvitamin D to optimal levels (greater than 30 ng/mL), thereby mitigating iatrogenic hypocalcemia and secondary hyperparathyroidism, and optimizing the effectiveness of an antiresorptive agent at mitigating skeletal-related events in prostate cancer patients, is examined in a 24-month study of adult male patients with bone-metastasized castration-resistant prostate cancer. In a randomized, double-blind controlled study, patients are treated with denosumab (120 mg every four weeks). All denosumab-treated patients are randomized to receive daily oral treatment with one softgel capsule containing either 30 mcg of 25-hydroxyvitamin D₃ in a modified release formulation or 400 IU of Vitamin D₃ in an immediate release formulation. A total of 500 subjects participate in this study, all of whom are aged 18 years or older with histologically confirmed prostate cancer. Prior to study admission, patients had to have received treatment for prostate cancer (e.g., bilateral orchiectomy or androgen-deprivation therapy for at least 6 months), have total serum testosterone lower than 50 ng/dL, and have three consecutive increasing PSA tests separated by at least 2 weeks with the last two PSA measurements greater than or equal to 1.0 μg/L. All patients have serum total 25-hydroxyvitamin D levels less than 30 ng/mL at the time of enrollment. All patients receive a radioisotope bone scan during screening with subsequent imaging by CT, MRI, or plain radiograph if needed to confirm bone metastases. All subjects receive calcium supplements (500 mg/day) and abstain from taking other Vitamin D supplements for 60 days before study start and continuing through study termination, and from significant sun exposure.

All subjects begin daily dosing with softgel capsules at the start of denosumab treatment. Serum total 25-hydroxyvitamin D, PTH, calcium, phosphorus, N- and C-telopeptides, and P1NP, and urinary calcium, phosphorus and creatinine, are measured monthly. Radiographic bone scans are conducted every 6 months to detect skeletal metastases, with a second imaging modality (CT, MRI, or plain radiograph) used to confirm diagnosis of any metastases detected. Bone mineral density at four sites (total hip, femoral neck, ⅓ radius and lumbar spine) is determined at the start of the study and thereafter at yearly intervals. After 3 months, the daily dosage of 25-hydroxyvitamin D₃ capsules is maintained unchanged in patients whose serum total 25-hydroxyvitamin D is between 50 and 90 ng/mL, and increased by one 30 mcg capsule in patients whose serum total 25-hydroxyvitamin D is below 50 ng/mL. The dosage is immediately lowered by one 30 mcg capsule per day in patients whose serum total 25-hydroxyvitamin D rises above 100 ng/mL or whose serum calcium is confirmed above 10.3 mg/dL.

After 6 months to 9 months, all subjects exhibit serum total 25-hydroxyvitamin D levels essentially stable in a range of 50 ng/mL to 90 ng/mL with 25-hydroxyvitamin D₃ treatment or between approximately 25 ng/mL to 35 ng/mL with Vitamin D₃ treatment. In patients treated with 25-hydroxyvitamin D₃, the incidence of hypocalcemia and severity of SHPT and hypercalcemia is markedly reduced once stable dosing has been achieved. In contrast, patients treated with Vitamin D₃ exhibit hypercalcemia and SHPT more frequently. After 24 months of treatment, the patients treated with denosumab and 25-hydroxyvitamin D₃ are found to have higher and more consistent serum levels of 25-hydroxyvitamin D₃ and lower serum PTH levels than patients treated with denosumab and vitamin D₃. Patients treated with denosumab and 25-hydroxyvitamin D₃ are found to have a significantly lower incidence of hypocalcemia, reduced plasma PTH levels and larger increases in bone mineral density and to have a significantly delayed time to first post-treatment SRE, compared to patients treated with denosumab and Vitamin D₃. Data from this study demonstrate that the modified release formulation of 25-hydroxyvitamin D₃ is effective at increasing serum 25-hydroxyvitamin D without causing unacceptable side effects related to calcium and PTH metabolism, and at mitigating hypocalcemia and augmenting the increases in bone mineral density and delayed time to first bone metastasis produced by denosumab.

### Example 8

Efficacy Study in Breast Cancer Patients

The effectiveness of oral modified-release 25-hydroxyvitamin D₃ in restoring serum total 25-hydroxyvitamin D to optimal levels (greater than 30 ng/mL), thereby mitigating hypocalcemia and SHPT and optimizing the effectiveness of denosumab at mitigating SRE in breast cancer patients, is examined in a 24-month study of adult female patients with breast cancer. In a randomized, double-blind controlled study, patients are treated with denosumab (120 mg every four weeks). All denosumab-treated patients are randomized to receive daily oral treatment with one softgel capsule containing either 30 mcg of 25-hydroxyvitamin D₃ in a modified release formulation or 400 IU of cholecalciferol in an immediate release formulation. All subjects participating in this study are aged 18 years or older with histologically or cytologically confirmed breast adenocarcinoma and current or prior radiographic (x-ray, CT or MRI) evidence of at least one bone metastasis. All subjects receive calcium supplements (500 mg/day) and abstain from taking other Vitamin D supplements for 60 days before study start and continuing through study termination, and from significant sun exposure. All subjects begin daily dosing with softgel capsules at the start of denosumab treatment. Serum total 25-hydroxyvitamin D, PTH, calcium, phosphorus, N- and C-telopeptides, and P1NP, and urinary calcium, phosphorus and creatinine, are measured monthly. Radiographic bone scans are conducted every 6 months to monitor skeletal metastases, with a second imaging modality (CT, MRI, or plain radiograph) used to confirm any metastases detected. Bone mineral density at four sites (total hip, femoral neck, ⅓ radius and lumbar spine) is determined at the start of the study and thereafter at yearly intervals. After 3 months, the daily softgel capsule dosage is maintained unchanged in patients whose serum total 25-hydroxyvitamin D is between 50 and 90 ng/mL and increased by one mcg capsule in patients whose serum total 25-hydroxyvitamin D is below 50 ng/mL. The dosage is immediately lowered by one capsule per day in patients whose serum total 25-hydroxyvitamin D rises above 100 ng/mL or whose serum calcium is confirmed above 10.3 mg/dL. After 6 to 9 months, the subjects' serum total 25-hydroxyvitamin D levels remain essentially stable with continued dosing, and rise to a level between about 50 ng/mL and about 90 ng/mL with 25-hydroxyvitamin D₃ treatment or to approximately 25 to 35 ng/mL with cholecalciferol treatment.

In patients treated with 25-hydroxyvitamin D₃, the incidence of hypocalcemia and severity of secondary hyperparathyroidism are markedly reduced once stable dosing has been achieved. However, in patients treated with vitamin D₃, hypocalcemia and secondary hyperparathyroidism occur more frequently. After 24 months of treatment, the patients treated with denosumab and 25-hydroxyvitamin D₃ are found to have higher and more consistent serum levels of 25-hydroxyvitamin D₃ and lower serum PTH levels than are patients treated with denosumab and vitamin D₃. Patients treated with denosumab and 25-hydroxyvitamin D₃ are found to have a significantly lower incidence of hypocalcemia and larger increases in bone mineral density and to have a significantly delayed time to additional bone metastasis, compared to patients treated with denosumab and Vitamin D₃. Data from this study demonstrate that the modified release formulation of 25-hydroxyvitamin D₃ is effective at increasing serum total 25-hydroxyvitamin D without causing unacceptable side effects related to calcium and PTH metabolism, and at mitigating hypocalcemia and augmenting the increases in bone mineral density and delayed time to bone metastasis produced by denosumab.

### Example 9

Safety Study in Patients with Metastatic Bone Disease Receiving Treatment with an Antiresorptive Agent

The safety and tolerability of oral modified release 25-hydroxyvitamin D₃ is examined in an open label, repeat-dose study of adult patients diagnosed with metastases in bone originating from breast or prostate cancer who are receiving ongoing treatment with denosumab or zoledronic acid for at least 3 months. At the start of the study, all patients have plasma PTH greater than 70 pg/mL as evidence of SHPT, serum calcium less than 9.8 mg/dL, spot urine Ca:Cr ratio ≤0.25 (≤250 mg/g creatinine) and an estimated glomerular filtration rate greater than 15 mL/min/1.73 m². Twenty-four (24) patients diagnosed with bone metastases subsequent to breast or pancreatic carcinoma are treated for up to 52 weeks with one or more capsules containing 30 mcg of 25-hydroxyvitamin D₃ in a modified release formulation. Denosumab or zoledronic acid are administered according to the typical standard of care for each patient's condition. Patients whose typical standard of care requires calcium and/or vitamin D supplementation receive less than 1000 mg/day of elemental calcium and/or 2000 IU/day or less of vitamin D (ergocalciferol and/or cholecalciferol). Patients do not receive any other vitamin D analogs (e.g., calcitriol, paricalcitol, doxercalciferol, etc.).

The 52-week study consists of a 40 week dose escalation phase followed by a 12-week maintenance phase. At the end of the maintenance phase, there is a two-week follow up phase. At the start of the study, all patients receive an initial daily dose of 30 mcg 25-hydroxyvitamin D₃, which is increased at four-week intervals over the course of the dose escalation phase up to a maximum daily dose of 300 mcg. The daily dose achieved by the end of the dose escalation study is the daily dose administered during the maintenance phase. Patients exhibiting a serum calcium level ≤10.3 mg/dL of the course of the study thus receive a daily dose of: 30 mcg 25-hydroxyvitamin D₃ at the start of the study; 60 mcg 25-hydroxyvitamin D₃ after 4 weeks; 90 mcg 25-hydroxyvitamin D₃ at 8 weeks; 120 mcg 25-hydroxyvitamin D₃ at 12 weeks; 150 mcg 25-hydroxyvitamin D₃ at 16 weeks; 180 mcg 25-hydroxyvitamin D₃ at 20 weeks; 210 mcg 25-hydroxyvitamin D₃ at 24 weeks; 240 mcg 25-hydroxyvitamin D₃ at 28 weeks; 270 mcg 25-hydroxyvitamin D₃ at 32 weeks; and 300 mcg 25-hydroxyvitamin D₃ at 36 weeks and through the maintenance phase. Patients exhibiting a serum calcium level exceeding 10.3 mg/dL for two consecutive visits will suspend dosing until serum calcium returns to ≤10.0 mg/dL, and then resume treatment at a reduced daily dose and enter a 12-week maintenance phase, followed by a 2-week follow-up period.

Blood samples are collected at 2-week intervals for monitoring serum levels of calcium and phosphorus. Samples are collected at 4-week intervals for monitoring plasma levels of PTH and PTHrP and serum total 25-hydroxyvitamin D, 24,25-dihydroxyvitamin D₃, calcitriol, and free and total calcifediol. Serum vitamin D metabolites and markers of bone metabolism, immune function, and tumor burden are measured at the beginning of the dose escalation phase and at the beginning and end of the maintenance phase. Urine samples are collected at 4-week intervals for monitoring the Ca/Cr ratio and urine chemistry. The genotype of vitamin D binding protein is determined for each subject at the beginning of the dose escalation phase.

Serum calcium gradually rises in the dose escalation phase while plasma PTH decreases. When plasma PTH is overly suppressed, serum calcium rises more quickly with continued dose escalation, increasing the risk of hypercalcemia. Patients exhibit significant increases in serum total 25-hydroxyvitamin D, 1,25-dihydroxyvitamin D, and 24,25-dihydroxyvitamin D, and decreases in plasma PTH. Patients receiving the starting dose level of 30 mcg of 25-hydroxyvitamin D₃ exhibit mean serum 25-hydroxyvitamin D levels of about 50 ng/mL. Patients receiving the dose level of 90 mcg of 25-hydroxyvitamin D₃ exhibit mean serum 25-hydroxyvitamin D levels of about 100 mg/mL. Patients receiving the highest dose level of 300 mcg of 25-hydroxyvitamin D₃ exhibit mean serum 25-hydroxyvitamin D levels of about 200 to about 300 ng/mL, for example, about 230 ng/mL. Data from this study demonstrate that a modified release formulation of 25-hydroxyvitamin D₃ is effective at increasing serum total 25-hydroxyvitamin D without causing unacceptable side effects related to calcium and PTH metabolism.

### Example 10

Efficacy Study in Patients with Metastatic Bone Disease Receiving Treatment with an Antiresorptive Agent

The effectiveness of oral modified-release 25-hydroxyvitamin D₃ in raising serum 25-hydroxyvitamin D and 1,25-dihydroxyvitamin D and delaying cancer progression is examined in a 6-month randomized, double-blind placebo-controlled study of adult patients diagnosed with metastases in bone originating from breast or prostate cancer who are receiving ongoing treatment with denosumab or zoledronic acid for at least 3 months. Patients are treated with one or more capsules containing 30 mcg of 25-hydroxyvitamin D₃ in a modified release formulation or placebo. Denosumab or zoledronic acid are administered according to the typical standard of care for each patient's condition. Patients whose typical standard of care requires calcium and/or vitamin D supplementation receive less than 1000 mg/day of elemental calcium and/or 2000 IU/day or less of vitamin D (ergocalciferol and/or cholecalciferol). Samples are collected at monthly intervals for monitoring serum and urine levels of calcium, plasma levels of PTH and serum total 25-hydroxyvitamin D. Serum markers of tumor burden and bone metabolism, as well as cancer progression are assessed at 3-month intervals.

Patients treated with 25-hydroxyvitamin D₃ are found to have a greater increase in serum calcium and decrease in plasma PTH, leading to reduced risk of hypocalcemia compared to patients receiving the placebo. Patients treated with denosumab or zoledronic acid and 25-hydroxyvitamin D exhibit an increased delay in time to additional bone metastasis, compared to patients receiving denosumab or zoledronic acid in combination with a placebo. Data from this study demonstrate that the modified release formulation of 25-hydroxyvitamin D₃ is effective at increasing serum total 25-hydroxyvitamin D 1,25-dihydroxyvitamin D and delaying cancer progression, without causing unacceptable side effects related to calcium and PTH metabolism.

### Example 11

Efficacy Study in Patients with Metastatic Bone Disease Receiving Treatment with an Antiresorptive Agent for Prevention of SREs.

The effectiveness of oral modified release 25-hydroxyvitamin D₃ in delaying the time to the first post-treatment SRE is examined in 24-month randomized, double-blind placebo-controlled studies of adult males with castration-resistant prostate cancer metastatic to bone or adult females with estrogen-independent breast cancer metastatic to bone, who are receiving ongoing treatment with denosumab or zoledronic acid for at least 3 months. Patients are treated with one or more capsules containing 30 mcg of 25-hydroxyvitamin D₃ in a modified release formulation or placebo. Denosumab or zoledronic acid are administered according to the typical standard of care for each patient's condition. Patients are monitored for SREs, including by appropriate non-invasive imaging techniques, and serum markers of tumor burden and bone metabolism at 3-month intervals, and at monthly intervals for serum and urine calcium levels and plasma PTH. Cancer progression is monitored at quarterly intervals.

Patients treated with 25-hydroxyvitamin D₃ are found to have a greater increase in serum calcium and decrease in plasma PTH, leading to reduced risk of hypocalcemia compared to patients receiving the placebo. Patients treated with denosumab or zoledronic acid and 25-hydroxyvitamin D exhibit an increased delay in time to additional bone metastasis or SRE, compared to patients receiving denosumab or zoledronic acid in combination with a placebo. Data from this study demonstrate that 25-hydroxyvitamin D₃ is effective at significantly increasing the observed time to a post-treatment SRE and inhibiting tumor progression compared to placebo.

The foregoing description has outlined, in general, the featured aspects of the invention. In reference to such, there is to be a clear understanding that the present invention is not limited to the method or detail of manufacture, chemical composition, or application of use described herein. Any other variation of manufacture, chemical composition, use, or application should be considered apparent as an alternative embodiment of the present invention. Other advantages and a fuller appreciation of the specific adaptations, compositional variations and chemical and physical attributes of this invention will be gained upon examination of the detailed description.

Also, it is understood that the phraseology and terminology used herein are for the purpose of description and should not be regarded as limiting. Throughout the specification and the claims which follow, unless the context requires otherwise, the use of “including,” “having,” and “comprising” and variations thereof herein is meant to encompass the stated integers and steps and equivalents thereof as well as additional items and equivalents thereof.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The foregoing description is given for clearness of understanding only, and no unnecessary limitations should be understood therefrom, as modifications within the scope of the invention may be apparent to those having ordinary skill in the art. All patents, publications and references cited herein are hereby fully incorporated by reference. In case of conflict between the present disclosure and incorporated patents, publications and references, the present disclosure should control.

What is claimed is:

1. A composition comprising 25-hydroxyvitamin D for use in a method of treatment of a patient, the method being:
i. a method of treating or preventing iatrogenic hypocalcemia and secondary hyperparathyroidism in a patient treated with an agent that increases the risk of hypocalcemia, where the method comprises administering to the patient an effective amount of said 25-hydroxyvitamin D; or
ii. a method of increasing bone mineral density in a patient treated with an agent that increases the risk of hypocalcemia, where the method comprises administering to the patient an effective amount of said 25-hydroxyvitamin D; or
iii. a method of decreasing the blood level of a bone resorption marker in a patient treated with an agent that increases the risk of hypocalcemia, where the method comprises administering to the patient an effective amount of said 25-hydroxyvitamin D; or
iv. a method of treating bone pain in a patient treated with an agent that increases the risk of hypocalcemia, where the method comprises administering to the patient an effective amount of said 25-hydroxyvitamin D; or
v. a method of increasing the time to the first post-treatment skeletal-related event in a patient treated with an agent that increases the risk of hypocalcemia, where the method comprises administering to the patient an effective amount of said 25-hydroxyvitamin D; or
vi. a method of treating a patient treated with an agent that increases the risk of hypocalcemia, where the method comprises administering to the patient an effective amount of said 25-hydroxyvitamin D to effectively and safely restore blood 25-hydroxyvitamin D levels to at least 30 ng/mL and to maintain blood 25-hydroxyvitamin D levels at such optimal levels.

2. A method of treating or preventing iatrogenic hypocalcemia and secondary hyperparathyroidism in a patient treated with an agent that increases the risk of hypocalcemia, comprising administering to the patient an effective amount of 25-hydroxyvitamin D.

3. A method of increasing bone mineral density in a patient treated with an agent that increases the risk of hypocalcemia, comprising administering to the patient an effective amount of 25-hydroxyvitamin D.

4. A method of decreasing the blood level of a bone resorption marker in a patient treated with an agent that increases the risk of hypocalcemia, comprising administering to the patient an effective amount of 25-hydroxyvitamin D.

5. A method of treating bone pain in a patient treated with an agent that increases the risk of hypocalcemia, comprising administering to the patient an effective amount of 25-hydroxyvitamin D.

6. A method of increasing the time to the first post-treatment skeletal-related event in a patient treated with an agent that increases the risk of hypocalcemia, comprising administering to the patient an effective amount of 25-hydroxyvitamin D.

7. A method of treating a patient treated with an agent that increases the risk of hypocalcemia, comprising administering to said patient an effective amount of 25-hydroxyvitamin D to effectively and safely restore blood 25-hydroxyvitamin D levels to at least 30 ng/mL and to maintain blood 25-hydroxyvitamin D levels at such optimal levels.

8. The composition for use or the method of any one of the preceding claims, wherein the patient treated with an agent that increases the risk of hypocalcemia is receiving or has previously received treatment with an agent that increases the risk of hypocalcemia.

9. The composition for use or the method of any one of the preceding claims, wherein the agent that increases the risk of hypocalcemia is selected from the group consisting of antiresorptive agents, anticonvulsant agents, corticosteroids, antihypercalcemia agents, antimicrobial agents, and combinations thereof

10. The composition for use or the method of any one of the preceding claims, wherein the agent that increases the risk of hypocalcemia is an antihypercalcemia agent, optionally the anti-hypercalcemia agent cinacalcet.

11. The composition for use or the method of claim 9 or 10, wherein the 25-hydroxyvitamin D is administered following administration of the antihypercalcemia agent.

12. The composition for use or the method of any one of claims 1-9, wherein the agent that increases the risk of hypocalcemia is an antiresorptive agent.

13. A composition comprising 25-hydroxyvitamin D for use in a method of treatment of a patient, the method being:
i. a method of lowering elevated serum parathyroid hormone levels in a patient having a bone metastasis and being treated with an antiresorptive agent, where the method comprises administering an effective amount of said 25-hydroxyvitamin D; or
ii. a method of stabilizing serum calcium levels in a patient having a bone metastasis and being treated with an antiresorptive agent.

14. A method of lowering elevated serum parathyroid hormone levels in a patient having a bone metastasis and being treated with an antiresorptive agent comprising administering an effective amount of 25-hydroxyvitamin D.

15. A method of stabilizing serum calcium levels in a patient having a bone metastasis and being treated with an antiresorptive agent.

16. The composition for use of claim 12 or 13 or the method of any one of claims 12 or 14-15, wherein the antiresorptive agent is selected from the group consisting of bisphosphonates, selective estrogen receptor modulators, calcitonin, hormones, monoclonal antibodies, and combinations thereof

17. The composition for use of claim 12 or 13 or 16, or the method of any one of claims 12 or 14-16, wherein the antiresorptive agent comprises a RANKL inhibitor, optionally the RANKL inhibitor denosumab.

18. The composition for use of claim 12 or 13 or 16, or the method of any one of claims 12 or 14-16, wherein the antiresorptive agent comprises a bisphosphonate, optionally the bisphosphonate zoledronic acid.

19. A composition comprising 25-hydroxyvitamin D for use in a method of treatment of a patient, the method being a method of treating hungry bone syndrome, where the method comprises administering an effective amount of said 25-hydroxyvitamin D to a patient in need of thereof.

20. A method of treating hungry bone syndrome comprising administering an effective amount of 25-hydroxyvitamin D to a patient in need of thereof.

21. The composition for use or method of any one of the preceding claims, wherein the patient has osteoporosis.

22. The composition for use or method of any one of the preceding claims, wherein the patient has cancer.

23. A composition comprising 25-hydroxyvitamin D for use in a method of treatment of a patient, the method being:
i. a method of managing iatrogenic hypocalcemia and secondary hyperparathyroidism in a patient with a bone metastasis receiving therapy with an antiresorptive agent, where the method comprises administering an effective amount of said 25-hydroxyvitamin D to prevent or reverse iatrogenic hypocalcemia and lower the patient's serum parathyroid hormone level; or
ii. a method of inhibiting the proliferation and migration of cancer cells, where the method comprises administering an effective amount of said 25-hydroxyvitamin D to a patient in need thereof; or
iii. a method of mitigating the progression of cancer in the bone, where the method comprises administering an effective amount of said 25-hydroxyvitamin D to a patient; or
iv. a method of treating a patient having cancer and a bone metastasis, where the method comprises the administration of (a) a prophylactic and continuing course of an effective amount of said 25-hydroxyvitamin D to stabilize serum 25-hydroxyvitamin D levels and calcium levels in said patient without causing or exacerbating hypercalcemia, followed by (b) treatment with an agent known to increase the risk of iatrogenic hypocalcemia, wherein the treatment in step (a) prevents and/or treats said iatrogenic hypocalcemia in said patient; or
v. a method of treating cancer in a patient, where the method comprises administering to the patient an effective amount of a combination of said 25-hydroxyvitamin D and an anticancer agent; or
vi. a method of mitigating cancer progression and/or a skeletal related event in a patient with a bone metastasis from a solid tumor, where the method comprises treating said patient with (a) an anticancer agent; (b) an antiresorptive agent and (c) said 25-hydroxyvitamin D compound, wherein the combination of (a), (b), and (c) is effective to slow tumor growth and/or metastasis and increase the time to the first post-treatment skeletal-related event.

24. A method of managing iatrogenic hypocalcemia and secondary hyperparathyroidism in a patient with a bone metastasis receiving therapy with an antiresorptive agent, comprising administering an effective amount of 25-hydroxyvitamin D to prevent or reverse iatrogenic hypocalcemia and lower the patient's serum parathyroid hormone level.

25. A method of inhibiting the proliferation and migration of cancer cells comprising administering an effective amount of 25-hydroxyvitamin D to a patient in need thereof.

26. A method of mitigating the progression of cancer in the bone comprising administering an effective amount of 25-hydroxyvitamin D to a patient.

27. A method of treating a patient having cancer and a bone metastasis comprising the administration of (a) a prophylactic and continuing course of an effective amount of 25-hydroxyvitamin D to stabilize serum 25-hydroxyvitamin D levels and calcium levels in said patient without causing or exacerbating hypercalcemia, followed by (b) treatment with an agent known to increase the risk of iatrogenic hypocalcemia, wherein the treatment in step (a) prevents and/or treats said iatrogenic hypocalcemia in said patient.

28. A method of treating cancer in a patient, comprising administering to the patient an effective amount of a combination of 25-hydroxyvitamin D and an anticancer agent.

29. A method of mitigating cancer progression and/or a skeletal related event in a patient with a bone metastasis from a solid tumor comprising treating said patient with (a) an anticancer agent; (b) an antiresorptive agent and (c) a 25-hydroxyvitamin D compound, wherein the combination of (a), (b), and (c) is effective to slow tumor growth and/or metastasis and increase the time to the first post-treatment skeletal-related event.

30. The composition for use of claim 23, option v or vi, or the method of claim 28 or 29, wherein the anticancer agent is selected from the group consisting of azacitidine, axathioprine, bevacizumab, bleomycin, capecitabine, carboplatin, chlorabucil, cisplatin, cyclophosphamide, cytarabine, daunorubicin, docetaxel, doxifluridine, doxorubicin, epirubicin, etoposide, fluorouracil, gemcitabine, herceptin, idarubicin, mechlorethamine, melphalan, mercaptopurine, methotrexate, mitoxantrone, oxaliplatin, paclitaxel, tafluposide, teniposide, tioguanine, retinoic acid, valrubicin, vinblastine, vincristine, vindesine, vinorelbine, receptor tyrosine kinase inhibitors, and combinations thereof.

31. The composition for use of claim 22 or 23, or the method of any one of claims 22 or 24-30, wherein the cancer is selected from the group consisting of bone cancer, bladder cancer, breast cancer, colon cancer, endometrial cancer, kidney cancer, leukemia, lung cancer, lymphoma, pancreatic cancer, prostate cancer, skin cancer, thyroid cancer, and metastatic forms thereof.

32. The composition for use or method of any one of the preceding claims, wherein the patient has a metastatic bone cancer, metastatic prostate cancer, metastatic lung cancer, and/or metastatic breast cancer.

33. The composition for use or method of any one of the preceding claims, wherein the patient has a bone tumor, optionally a bone metastasis from a solid tumor.

34. The composition for use or method of any one of the preceding claims, wherein the patient has impaired renal function, optionally associated with Chronic Kidney Disease Stage 1, 2, 3, 4, or 5.

35. The composition for use or method of any one of the preceding claims, wherein the effective amount of 25-hydroxyvitamin D is effective to restore or maintain the patient's serum calcium level to at least about 8.0 mg/dL, optionally in a range of about 8.3 mg/dL to about 11.6 mg/dL.

36. The composition for use or method of any one of the preceding claims, wherein the effective amount of 25-hydroxyvitamin D is effective to safely increase the patient's serum level of 25-hydroxyvitamin D to at least 30 ng/mL, optionally in a range of about 30 ng/mL to about 100 ng/mL.

37. The composition for use or method of any one of the preceding claims, wherein the effective amount of 25-hydroxyvitamin D is effective to decrease the patient's serum parathyroid hormone level, optionally by 30% or more.

38. The composition for use or method of any one of the preceding claims, wherein the effective amount of 25-hydroxyvitamin D is effective to decrease the patient's serum parathyroid hormone related peptide levels.

39. The composition for use or method of any one of the preceding claims, wherein the effective amount of 25-hydroxyvitamin D is effective to safely increase the patient's serum level of 1,25-dihydroxyvitamin D to above the normal range, optionally at least to 50 pg/mL.

40. The composition for use or method of any one of the preceding claims, wherein the effective amount of 25-hydroxyvitamin D is effective to achieve or maintain safe serum phosphorus levels and treat or prevent hypophosphatemia.

41. The composition for use or method of any one of the preceding claims, wherein the effective amount of 25-hydroxyvitamin D is effective to have a positive effect on the patient's serum level of a marker of bone formation by more than what is accomplished with treatment with the antiresorptive agent alone.

42. The composition for use or method of any one of the preceding claims, wherein the effective amount of 25-hydroxyvitamin D is effective to decrease the patient's serum level of a marker of bone resorption, optionally by at least 20%.

43. The composition for use or method of claim 42, wherein the marker of bone resorption is selected from the group consisting of PTHrP, FGF23, NTX, CTX, TRAC-5b, and combinations thereof.

44. The composition for use or method of any one of the preceding claims, wherein the effective amount of 25-hydroxyvitamin D is effective to maintain or decrease the patient's tumor burden.

45. The composition for use or method of claim 44, wherein the effective amount of 25-hydroxyvitamin D is effective to maintain or decrease the patient's serum level of a marker of tumor burden.

46. The composition for use or method of claim 45, wherein the marker of tumor burden is selected from the group consisting of CEA, CA 125, CA15-3, CA 27-29, prostate specific antigen (PSA), and combinations thereof.

47. The composition for use or method of any one of the preceding claims, wherein the effective amount of 25-hydroxyvitamin D is effective to inhibit the proliferation and/or migration of cancer cells.

48. The composition for use or method of any one of the preceding claims, wherein the 25-hydroxyvitamin D is administered in a modified release formulation.

49. The composition for use or method of claim 48, wherein the modified release formulation is selected from the group consisting of a sustained release formulation, a delayed release formulation, and a delayed, sustained release formulation.

50. The composition for use or method of claim 48 or 49, wherein the modified release formulation comprises a waxy formulation.

51. The composition for use or method of any one of claims 48-50 wherein the modified release formulation consists essentially of about 20 wt % paraffin, about 20 wt % to about 25 wt % glycerol monostearate, about 10 wt % a mixture of lauroyl macrogolglycerides and lauroyl polyoxylglycerides, about 30 wt % to about 35 wt % mineral oil, and about 10 wt % to about 15 wt % hydroxyl propyl methylcellulose.

52. The composition for use or method of any one of the preceding claims, wherein the effective amount of 25-hydroxyvitamin D is administered in an oral sustained release formulation.

53. The composition for use or method of any one of the preceding claims, wherein the 25-hydroxyvitamin D is administered in the form of a capsule.

54. The composition for use or method of any one of the preceding claims, wherein the effective amount of 25-hydroxyvitamin D is absorbed primarily in the ileum and/or colon of the patient.

55. The composition for use or method of any one of claims 1-47, wherein the 25-hydroxyvitamin D is administered in an oral immediate release formulation in multiple daily doses in order to produce a pharmacokinetic profile of serum 25-hydroxyvitamin D that is similar to that achieved by an oral modified or sustained release formulation.

56. The composition for use or method of any one of claims 1-49, wherein the 25-hydroxyvitamin D is administered in a sterile intravenous formulation.

57. The composition for use or method of claim 56, wherein the 25-hydroxyvitamin D is administered in an intravenous bolus.

58. The composition for use or method of claim 57, wherein the 25-hydroxyvitamin D is infused over a course of at least 1 hour.

59. The composition for use or method of any one of the preceding claims, wherein the 25-hydroxyvitamin D is selected from the group consisting of 25-hydroxyvitamin D₂, 25-hydroxyvitamin D₃, 25-hydroxyvitamin D₄, 25-hydroxyvitamin Ds, 25-hydroxyvitamin D₇ and combinations thereof.

60. The composition for use or method of any one of the preceding claims, wherein the 25-hydroxyvitamin D comprises 25-hydroxyvitamin D₃ and/or 25-hydroxyvitamin D₂.

61. The composition for use or method of any one of the preceding claims, wherein the 25-hydroxyvitamin D comprises 25-hydroxyvitamin D₃.

62. The composition for use or method of any of the preceding claims, wherein the 25-hydroxyvitamin D is administered in a dosage of 30 μg to 300 μg per day.

63. Use of 25-hydroxyvitamin D, optionally in a modified release formulation, as adjunctive therapy to treat or prevent hypocalcemia in a patient in need thereof.

64. A composition comprising 25-hydroxyvitamin D, optionally in a modified release formulation, for use in a method of treatment of a patient, the method being an adjunctive therapy to treat or prevent hypocalcemia in a patient in need thereof.

65. A pharmaceutical composition comprising (a) 25-hydroxyvitamin D and (b) an agent that increases the risk of hypocalcemia and/or an anticancer agent.

66. A kit comprising (a) 25-hydroxyvitamin D, (b) an agent that increases the risk of hypocalcemia and/or an anticancer agent, and (c) instructions for co-administering effective amounts of (a) and (b) to a patient in need thereof.
